(19) United States
(12) Patent Application Publication
(10) Pub. No.: US 2024/0408047 A1
(43) Pub. Date: Dec. 12, 2024

(54) HYPOXIA INDUCIBLE FACTOR-2(ALPHA) INHIBITORS FOR THE TREATMENT OF BLADDER CANCER

(71) Applicant: NIKANG THERAPEUTICS, INC., Wilmington, DE (US)

(72) Inventor: Jing LU, Wilmington, DE (US)

(21) Appl. No.: 18/700,682

(22) PCT Filed: Oct. 17, 2022
(86) PCT No.: PCT/US2022/046922

§ 371 Date: Apr. 11, 2024

Related U.S. Application Data
(60) Provisional application No. 63/257,069, filed on Oct. 18, 2021.

(51) Int. Cl. A61K 31/277; A61K 31/282; A61K 31/337; A61K 31/44; A61K 31/47; A61K 31/502; A61K 31/506; A61K 31/519; A61K 31/704; A61K 33/243; A61K 39/395; A61P 35/00

(52) U.S. Cl. CPC A61K 31/277; A61K 31/282; A61K 31/337; A61K 31/44; A61K 31/47; A61K 31/502; A61K 31/506; A61K 31/519; A61K 31/704; A61K 33/243; A61K 39/3955; A61P 35/00

(57) ABSTRACT

The present disclosure is directed method for the treatment of bladder cancer with Hypoxia Inducible Factor 2α (HIF-2α) inhibitors. Also disclosed are pharmaceutical compositions comprising the same.

### CROSS-REFERENCE TO RELATED APPLICATIONS

This application claims the benefit of U.S. Provisional Application No. 63/257,069, filed on Oct. 18, 2021, the contents of which is herein incorporated by reference in its entirety.

### FIELD OF THE DISCLOSURE

The present disclosure is directed to methods for the treatment of bladder cancer with Hypoxia Inducible Factor 2α (HIF-2α) inhibitors. Also disclosed are pharmaceutical compositions comprising the same.

### BACKGROUND

Bladder cancer (BC) is a major source of morbidity and mortality, with an estimated 500,000 new cases and 200,000 deaths worldwide each year (Lenis et al. (2020) JAMA 324(19):1980-1991; hereinafter “Lenis et al.”). In the US alone, there are more than 80,000 new cases and 17,000 deaths projected in 2021.

Bladder cancer is likely to arise from different cells along the papillary/luminal and nonpapillary/basal tracks of the urinary bladder. It is classified, based on standardized histopathology features as described by the World Health Organization, into non-muscle-invasive bladder cancer (NMIBC, when tumors are confined to the urothelium and the lamina propria) and muscle-invasive bladder cancer (MIBC, when tumors invade muscle and beyond).

NMIBC represents approximately 70% to 80% of organ-confined bladder cancer. NMIBC is commonly treated by transurethral tumor resection (TUR) with and without adjuvant intravesical instillations (Babjuk et al. (2019) Eur Urol 76(5):639-57). Although rarely life-threatening, NMIBC has a propensity for recurrence and progression to muscle invasive disease. Therefore, better and novel therapeutics with different mechanism(s) may provide improved treatment outcomes in NMIBC.

In contrast, MIBC has a high tendency for metastatic spread and drive the most morbidity and mortality eventually. While early diagnosis and multimodality therapy results in improved outcomes for patient with non-metastatic MIBC, metastatic disease remains generally incurable, with a relative 5-year overall survival (OS) rate of 15% (Nawaz and Webster (2016) Nat Rev Drug Discov 15(9):599-600). Systemic combination chemotherapy is the standard approach for the initial treatment of patients with inoperable locally advanced or metastatic MIBC. Although initial response rates are high, the durability of response is variable with the median survival upon multiagent chemotherapy at approximately 15 months, and the prognosis of patients with advanced disease remains poor.

The high mutational burden of bladder cancer renders it susceptible to immunotherapy, particularly with checkpoint inhibitors targeting programmed cell death-1 (PD-1) and its ligand, PD-L1. Five checkpoint inhibitors have been approved for the treatment of bladder cancer at various stages of disease to date, including Pembrolizumab, Atezolizumab, Nivolumab, Durvalumab and Avelumab. Despite the success of immune checkpoint inhibitor (ICI) monotherapy, long-term durable response rates remain low, and most patients relapse (Nadal and Bellmunt (2019) Cancer Treat Rev. 76:10-21, hereinafter “Nadal and Bellmunt”; Lenis et al.; Nadal et al. (2021) Hematol Oncol Clin North Am. 35(3):469-93; hereinafter “Nadal et al.”).

Recent advances in pathophysiology and molecular biology understanding of bladder cancer also led to the approval of two novel targeted therapeutics, a small molecule FGFR inhibitor and an antibody-drug conjugate. Although FGFR alterations are more frequent in non-MIBC, they are also found in up to 21% of locally advanced or metastatic bladder cancer, with amplifications, mutations, and fusions in FGFR gene. Erdafitinib, a pan-FGFR inhibitor, is approved for patients with FGFR2 and FGFR3-altered advanced bladder cancer, with the first PCR-based diagnostic companion to detect FGFR alterations in the tissue of patients simultaneously approved (Alifrangis et al. (2019) Nat Rev Urol 16:465-83; Nadal and Bellmunt; Nadal et al.). Antibody-drug conjugates exploit the highly expressed nectin-4 protein as the target for drug delivery. Enfortumab vedotin, which uses an anti-nectin-4 antibody linked to the microtubule-disrupting molecule monomethyl auristatin E, demonstrated an objective response rate of 44% among patients who progressed following treatment with chemotherapy and immunotherapy. This led to FDA approval of enfortumab vedotin in this dual-refractory setting (Nadal and Bellmunt; Lenis et al.).

Despite the recent approval of multiple immune and targeted therapies, there still remains a need for novel therapeutic agents for the treatment of bladder cancer in both monotherapy and combination therapy settings.

### SUMMARY

Disclosed herein are methods for treating bladder cancer by inhibition of HIF-2α. The methods are based, at least in part, on results obtained upon treatment of certain bladder cancer xenograft models, including MIBC T24 model, with 3-fluoro-5-(((1S,2aR)-1,3,3,4,4-pentafluoro-2a-hydroxy-2,2a,3,4-tetrahydro-1H-cyclopenta[cd]inden-7-yl)oxy)benzonitrile (hereinafter referred to as Compound 1). Compound 1 is a potent and selective HIF-2α small molecule inhibitor and is disclosed in Table 1, as compound No. 5, of PCT Application No. Publication No. WO 2020/214853.

Compound 1 was tested in bladder cancer xenograft models, NMIBC models RT112 and RT4 and a MIBC T24 model. Treatment with 20 mg/kg of Compound 1 resulted in significant anti-tumor effect (TGI=44%) in the RT4 model and a complete regression in the T24 model (FIG. 1). The complete tumor regression in MIBC T24 tumor model by Compound 1 is unexpected as both HIF-1α and HIF-2α could be detected under normoxia condition (20% 02) and were upregulated under hypoxic condition (1% O₂) in T24 cells (FIG. 2(a)).

HIF-1α and HIF-2α are transcription factors that mediate the cellular response to hypoxia and facilitate tumor growth. Since HIF-1α and HIF-2α have been found to share functional redundancy in many cellular settings, in order to determine whether one or both of HIF-1α and HIF-2α are drivers of turmorgensis in in T24 cells, the following were determined: mRNA levels of vascular endothelial growth factor A (VEGFA) and glucose transporter 1 (GLUT1) genes in response to hypoxia, siRNA knowndown of HIF-1α and HIF-2α, and treatment with Compound 1. VEGFA and GLUT1 are key hypoxia-downstream genes that regulate tumor angiogenensis and metabolic reprogramming, thereby promoting tumor growth. It was discovered, that while both HIF-1α and HIF-2α were upregulated in T24 cells under hypoxia conditions (FIG. 2(a)), siRNA knockdown of HIF-2α and treatment of the T24 cells with Compound 1 significantly suppressed hypoxia-induced upregulation of VEGFA and GLUT1 (FIGS. 2(b) and 2(c)), respectively. In contrast, HIF-1α knockdown upregulated VEGFA and GLUT1 mRNA levels (FIG. 2(a)). These results establish that in the T24 cancer, HIF-2α, rather than HIF-1α, regulates hypoxia responses critical to T24 bladder cancer growth. Therefore, HIF-2α inhibitors have the potential to be effective for the treatment of bladder cancer either alone or in combination with other anti-cancer agents and/or radiation therapy.

In a first aspect, provided is a method of treating bladder cancer in a patient, comprising administering to the patient in need thereof, a therapeutically effective amount of a HIF-2α inhibitor or a pharmaceutically acceptable salt thereof, in a pharmaceutical composition comprising the HIF-2α inhibitor or a pharmaceutically acceptable salt thereof and a pharmaceutically acceptable excipient.

In a first embodiment of the first aspect, the HIF-2α inhibitor is:
(a) a compound of Formula (I):

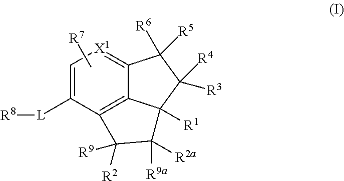

wherein:
X¹ is CH or N;
R¹ is hydroxy, halo, amino, —OP(O)(OH)₂, —OCH₂OP(O)(OH)₂, —OCOR¹⁰, —OCOOR¹¹, —OCONR¹²R¹³, —OCHR¹⁴OCOR¹⁵ or —OCHR¹⁴OCOOR^{15a} where R¹⁰, R¹¹, R¹⁵, and R^{15a} are independently alkyl or alkyl substituted with amino, carboxy or hydroxy, R¹² and R¹³ are independently hydrogen, alkyl, or alkyl substituted with amino, carboxy or hydroxy or R¹² and R¹³ together with the nitrogen atom to which they are attached form optionally substituted heterocyclyl, and each R¹⁴ is hydrogen, alkyl, or haloalkyl;
R² is hydrogen, deuterium, alkyl, halo, haloalkyl, alkenyl, or alkynyl;
R^{2a} is hydrogen, halo, or deuterium;
R³ and R⁴ are independently hydrogen, deuterium, alkyl, cycloalkyl, halo, haloalkyl, hydroxyalkyl, or alkoxyalkyl; or R³ and R⁴ together with the carbon to which they are attached form oxo, 3 to 6 membered cycloalkylene, or 4 to 6 membered optionally substituted heterocyclylene;
R⁵ is hydrogen, deuterium, alkyl, halo, haloalkyl, hydroxy, or alkoxy;
R⁶ is hydrogen, deuterium, alkyl, cycloalkyl, or halo; or
R⁵ and R⁶ together with the carbon to which they are attached form oxo, alkyldienyl, 3 to 6 membered cycloalkylene, or 4 to 6 membered optionally substituted heterocyclylene; provided R⁵ and R⁶ and R³ and R⁴ together with the carbon to which they are attached do not form oxo, cycloalkylene or optionally substituted 4 to 6 membered heterocyclylene simultaneously;
R⁷ is hydrogen, deuterium, alkyl, alkoxy, cyano, halo, haloalkyl, or haloalkoxy;
L is a bond, S, SO, SO₂, O, CO, or NR¹⁶ where R¹⁶ is hydrogen or alkyl;
R⁸ is alkyl, haloalkyl, hydroxyalkyl, alkoxyalkyl, aminoalkyl, cycloalkyl, cycloalkenyl, bicyclic cycloalkyl, fused phenyl, oxocycloalkenyl, cycloalkylalkyl, aryl, aralkyl, heterocyclyl, spirocycloalkyl, spiroheterocyclyl, heterocyclylalkyl, heteroaryl, or heteroaralkyl wherein aryl or heteroaryl, each by itself or as part of aralkyl or heteroaralkyl, heterocyclyl by itself or as part of heterocyclylalkyl and fused phenyl are substituted with R^{a}, R^{b}, R^{c}, R^{g} and R^{h} wherein R^{a}, R^{b}, and R^{c} are independently selected from hydrogen, deuterium, alkyl, haloalkyl, haloalkyloxy, alkoxy, hydroxy, halo, cyano, hydroxyalkyl, alkoxyalkyl, aminoalkyl, alkenyl, alkynyl, alkylidenyl, optionally substituted aryl, optionally substituted heteroaryl, and optionally substituted heterocyclyl and R^{g} and R^{h} are independently selected from hydrogen, deuterium, and halo;
R⁹ is hydrogen, alkyl, cycloalkyl, hydroxy, alkoxy, cyano, halo, haloalkyl, haloalkoxy, alkylsulfoxide, alkylsulfonyl, or heteroaryl wherein the heteroaryl is optionally substituted with R^{d}, R^{e}, and R^{f} independently selected from hydrogen, alkyl, haloalkyl, haloalkoxy, alkoxy, hydroxy, halo, and cyano; or
when R⁹ and R² are attached to the same carbon atom, they can combine to form oxo, alkyldienyl, 3 to 6 membered cycloalkylene, or 4 to 6-membered heterocyclylene; and
R^{9a} is hydrogen, halo, or deuterium;
(b) 3-(((1S,2S,3R)-2,3-difluoro-1-hydroxy-7-(methylsulfonyl)-2,3-dihydro-1H-inden-4-yl)oxy)-5-fluorobenzonitrile;
(c) NovartisDFF332; or
(d) Arcus AB521;
   or a pharmaceutically acceptable salt thereof.

In a second embodiment of the first aspect, the HIF-2 inhibitor is a compound of Formula (II) or (III):

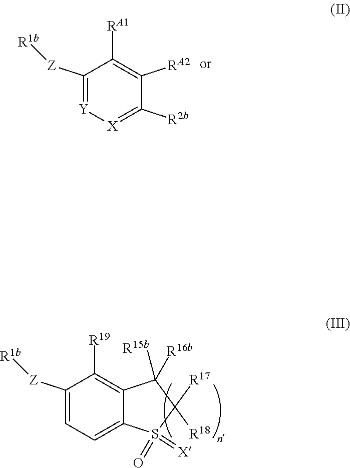

where Formula (II) and (III) correspond to the formulas numbered (I′) and (II), respectively, in PCT Application publication No. WO 2019/191227; and where X, Y, Z, R^{1b}, R^{A1}, R^{A2}, and R^{2b} of Formula (II) and n′, Z, X′, R¹, R^{15b}, R^{16b}, R¹⁷, R¹⁸, and R¹⁹ of Formula (III) are as defined in paragraphs [008], [024], and [033] to [049] of PCT Application publication No. WO 2019/191227, and these paragraphs are incorporated herein by reference in their entireties. Embodiments of compounds of Formula (II) and (III) are disclosed in paragraphs [[009] to [023], [025] to [026] and [0125] to [0187] and disclosed as specific compounds 1 to 833 (Table 1) and II-1 to II-60 (Table 2) of PCT Application publication No. WO 2019/191227, and these paragraphs and specific compounds are also incorporated herein by reference in their entireties.

In a third embodiment of the first aspect, the HIF-2 inhibitor is a compound of Formula (IV):

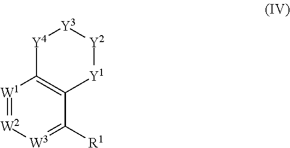

where Formula (IV) corresponds to formula (I) in PCT Application publication No. WO 2021/188769; and where Y¹, Y², Y³, Y⁴, W¹, W², W³, and R¹ of Formula (IV) and embodiments thereof (i.e., compounds of formulas II, III, (IV-a) to (IV-f), and (V-a) to (V-g), as numbered in WO 2021/188769) in paragraphs [0055], [0058], [0059] to [0089] and definitions of terms used in such formulae in paragraphs [0012] to [0051] are disclosed in PCT Application publication No. WO 2021/188769, and these formulae and paragraphs are incorporated herein by reference in their entireties. The specific compounds 1 to 248 disclosed in Tables 1, 2 and 3 of PCT Application publication No. WO 2021/188769, are also incorporated herein by reference in their entireties.

In a fourth embodiment of the first aspect, the HIF-2 inhibitor is (S)-1′-chloro-8-(difluoromethoxy)-8′,8′-difluoro-6-(trifluoromethyl)-7′,8′-dihydro-3H,6′H-spiro[imidazo[1,2-a]-pyridine-2,5′-isoquinoline] or a pharmaceutically acceptable salt thereof (DFF332).

In a fourth aspect, provided is a compound of Formula (I), (II), (III), (IV), including Arcus AB521, or Novartis DFF332; (or any of the embodiments thereof described herein), or a pharmaceutically acceptable salt thereof, for use in the treatment of bladder cancer.

In a fifth aspect, provided is a compound of Formula (I), (II), (III), (IV), including Arcus AB521, or Novartis DFF332; (or any of the embodiments thereof described herein), or a pharmaceutically acceptable salt thereof for preparation of a medicament for use in the treatment of bladder cancer.

In a sixth aspect, the method of any one of the aforementioned aspects is wherein the compound disclosed therein can be administered in combination with one or more anti-cancer agents disclosed in this Application, optionally with radiotherapy and/or surgery. In an embodiment of the fifth aspect, the one or more anti-cancer agent is selected from an FGFR inhibitor (such as vofatamab, infigratinib, LY2874455 ((R,E)-2-(4-(2-(5-(1-(3,5-dichloropyridin-4-yl)ethoxy)-1H-indazol-3-yl)vinyl)-1H-pyrazol-1-yl)ethan-1-ol), pemigatinib, rogaratinib, PRN1371 (8-(3-(4-acryloylpiperazin-1-yl)propyl)-6-(2,6-dichloro-3,5-dimethoxyphenyl)-2-(methylamino)pyrido[2,3-d]pyrimidin-7(8H)-one), zoligratinib, derazantinib, zoligratinib [Debio-1347, (5-amino-1-(2-methyl-1H-benzo[d]imidazol-6-yl)-1H-pyrazol-4-yl)(1H-indol-2-yl)methanone], and erdafitinib), a checkpoint inhibitor (such as a PD1, PDL1, or CTLA-4 inhibitor (such as nivolumab, pembrolizumab, pidilizumab, MEDI-0680 (a humanized IgG4κ anti-programmed cell death-1 antibody), durvalumab, BMS-936559 (an anti-PD-L1 monoclonal antibody), cetrelimab, avelumab, ipilimumab, tremelimumab, and atezolizumab)), afatinib, lapatinib, erlotinib, pertuzumab, trastuzumab, trastuzumab deruxtecan, bevacizumab, ramucirumab, sorafenib, lenvatinib, carbonzanitib, pazopanib, olaparib, AZD1775 (threonylthreonyltyrosylalanylaspartylphenylalanylisoleucylalanylserylglycylarginylthreonylglycy larginylarginylasparaginylalanylisoleucylhistidylaspartic acid), vistusertib, Linrodostat, Cisplatin, Carboplatin, Doxorubicin, Enfortumab Vedotin-ejfv, Mitomycin, Sacituzumab Govitecan-hziy, RC48-ADC (disitamab vedotin), Thiotepa, valrubicin, gemcitabine, methotrexate, vinblastine, docetaxel, paclitaxel, pemetrexed, Bacillus Calmette-Guerin, and Interferon.

In a seventh aspect, provided is a compound of formula (V):

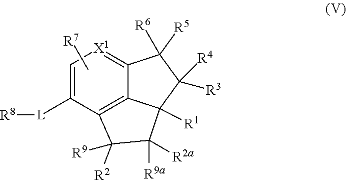

wherein:
X¹ is CH or N;
R¹ is hydroxy, halo, amino, —OP(O)(OH)₂, —OCH₂OP(O)(OH)₂, —OCOR¹⁰, —OCOOR¹¹, —OCONR¹²R¹³, —OCHR¹⁴OCOOR¹⁵ or —OCHR¹⁴OCOOR^{15a} where R¹⁰, R¹¹, R¹⁵, and R^{15a} are independently alkyl or alkyl substituted with amino, carboxy or hydroxy, R¹² and R¹³ are independently hydrogen, alkyl, or alkyl substituted with amino, carboxy or hydroxy or R¹² and R¹³ together with the nitrogen atom to which they are attached form optionally substituted heterocyclyl, and each R¹⁴ is hydrogen, alkyl, or haloalkyl;
R² is hydrogen, deuterium, alkyl, halo, haloalkyl, alkenyl, or alkynyl;
R^{2a} is hydrogen, halo, or deuterium;
R³ and R⁴ are independently hydrogen, deuterium, alkyl, cycloalkyl, halo, haloalkyl, hydroxyalkyl, or alkoxyalkyl; or
R³ and R⁴ together with the carbon to which they are attached form oxo, 3 to 6 membered cycloalkylene, or 4 to 6 membered optionally substituted heterocyclylene;
R⁵ is hydrogen, deuterium, alkyl, halo, haloalkyl, hydroxy, or alkoxy;
R⁶ is hydrogen, deuterium, alkyl, cycloalkyl, or halo; or
R⁵ and R⁶ together with the carbon to which they are attached form oxo, alkyldienyl, 3 to 6 membered cycloalkylene, or 4 to 6 membered optionally substituted heterocyclylene; provided R⁵ and R⁶ and R³ and R⁴ together with the carbon to which they are attached do not form oxo, cycloalkylene or optionally substituted 4 to 6 membered heterocyclylene simultaneously;
R⁷ is hydrogen, deuterium, alkyl, alkoxy, cyano, halo, haloalkyl, or haloalkoxy;
L is a bond, S, SO, SO₂, O, CO, or NR¹⁶ where R¹⁶ is hydrogen or alkyl;
R⁸ is fused phenyl substituted with R^{a}, R^{b}, R^{c}, R^{g} and R^{h} wherein R^{a}, R^{b}, and R^{c} are independently selected from hydrogen, deuterium, alkyl, haloalkyl, haloalkyloxy, alkoxy, hydroxy, halo, cyano, hydroxyalkyl, alkoxyalkyl, aminoalkyl, alkenyl, alkynyl, alkylidenyl, optionally substituted aryl, optionally substituted heteroaryl, and optionally substituted heterocyclyl and R^{g} and R^{h} are independently selected from hydrogen, deuterium, and halo;
R⁹ is hydrogen, alkyl, cycloalkyl, hydroxy, alkoxy, cyano, halo, haloalkyl, haloalkoxy, alkylsulfoxide, alkylsulfonyl, or heteroaryl wherein the heteroaryl is optionally substituted with R^{d}, R^{e}, and R^{f} independently selected from hydrogen, alkyl, haloalkyl, haloalkoxy, alkoxy, hydroxy, halo, and cyano; or
when R⁹ and R² are attached to the same carbon atom, they can combine to form oxo, alkyldienyl, 3 to 6 membered cycloalkylene, or 4 to 6-membered heterocyclylene; and
R^{9a} is hydrogen, halo, or deuterium;
   or a pharmaceutically acceptable salt thereof.

In certain embodiments of the seventh aspect, L is a bond and the groups R¹, R², R³, R⁴, R⁵, R⁶, R⁷, R⁹, R^{2a}, R^{9a} are as defined in the embodiments 2a to 25 and 31 to 37 herein below.

### BRIEF DESCRIPTION OF THE DRAWINGS

FIG. 1 provides anti-tumor effect of HIF-2α inhibitor Compound 1, Form A polymorph, in mice bladder cancer Xenograft models RT112, RT4, and T24.

FIG. 2(a) provides induction of HIF-1α and HIF-2α protein levels in bladder cancer cells T24 under normoxic and hypoxic conditions at 72 h.

FIG. 2(b) shows effects on hypoxia-induced upregulation of GLUT1 and VEGFA in bladder cancer T24 cells treated with siRNA of HIF-1α, HIF-2α or control under normoxia or hypoxia conditions at 72 h.

FIG. 2(c) shows effects on hypoxia-induced upregulation of GLUT1 and VEGFA in bladder cancer T24 cells treated with 1 μM Compound 1 or DMSO under normoxia or hypoxia conditions at 72 h.

FIG. 3 depicts a representative XRPD diffractogram of crystalline Form A polymorph of Compound 1.

### DETAILED DESCRIPTION

### Definitions

Unless otherwise stated, the following terms used in the specification and claims, including the terms X¹, R¹, R², R^{2a}, R³, R⁴, R⁵, R⁶, R⁷, R⁸, R⁹, R^{9a} in the compound of Formula (I) and (V), are defined for the purposes of this Application and have the following meaning unless stated otherwise.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear monovalent hydrocarbon radical of two to six carbon atoms or a branched monovalent hydrocarbon radical of three to six carbon atoms containing a double bond, e.g., propenyl, butenyl, and the like.

“Alkyldienyl” is alkenyl as defined above that is attached via a terminal divalent carbon. For example, in the compound below:

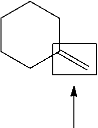

the alkyldienyl group is enclosed by the box which is indicated by the arrow.

“Haloaykdienyl” is alkyldienyl that is substituted with one or two halo, each group as defined herein.

“Alkynyl” means a linear monovalent hydrocarbon radical of two to six carbon atoms or a branched monovalent hydrocarbon radical of three to six carbon atoms containing a triple bond, e.g., propynyl, butynyl, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Alkylsulfoxide” means a —SOR radical where R is alkyl as defined above, e.g., methylsulfoxide, ethylsulfoxide, and the like.

“Amino” means a —NH₂.

“Alkylamino” means a —NHR radical where R is alkyl as defined above, e.g., methylamino, ethylamino, propylamino, or 2-propylamino, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen, alkyl, haloalkyl, hydroxyalkyl, alkoxyalkyl, or alkylcarbonyl, each as defined herein, e.g., aminomethyl, aminoethyl, methylaminomethyl, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, such as one or two alkoxy groups, as defined above, e.g., 2-methoxyethyl, 1-, 2-, or 3-methoxypropyl, 2-ethoxyethyl, and the like.

“Alkoxycarbonyl” means a —C(O)OR radical where R is alkyl as defined above, e.g., methoxycarbonyl, ethoxycarbonyl, and the like.

“Alkylcarbonyl” means a —C(O)R radical where R is alkyl as defined herein, e.g., methylcarbonyl, ethylcarbonyl, and the like.

“Aryl” means a monovalent monocyclic or bicyclic aromatic hydrocarbon radical of 6 to 10 ring atoms e.g., phenyl or naphthyl.

“Aralkyl” means a -(alkylene)-R radical where R is aryl as defined above, e.g., benzyl, phenethyl, and the like.

“Bicyclic cycloalkyl” means a fused bicyclic saturated monovalent hydrocarbon radical of six to ten carbon atoms which is optionally substituted with one or two substituents independently selected from alkyl, halo, alkoxy, hydroxy, and cyano. Examples include, but are not limited to, decalin, octahydro-1H-indene, and the like.

“Cycloalkyl” means a monocyclic saturated monovalent hydrocarbon radical of three to ten carbon atoms optionally substituted with one or two substituents independently selected from alkyl, alkyldienyl, halo, alkoxy, hydroxy, cyano, haloalkyldienyl and cyanoalkyl. Examples include, but are not limited to, cyclopropyl, cyclobutyl, cyclopentyl, cyclohexyl, 1-cyanocycloprop-1-yl, 1-cyanomethylcycloprop-1-yl, 3-fluorocyclohexyl, and the like. Cycloalkyl may include cycloalkylene as defined herein.

“Cycloalkylalkyl” means a -(alkylene)-R radical where R is cycloalkyl as defined above, e.g., cyclopropylmethyl, cyclohexylmethyl, and the like.

“Cycloalkylene” means a divalent cycloalkyl, as defined above, unless stated otherwise.

“Cycloalkenyl” means a monocyclic monovalent hydrocarbon radical of three to ten carbon atoms containing one or two double bond(s) optionally substituted with one or two substituents independently selected from alkyl, halo, alkoxy, hydroxy, cyano, and cyanoalkyl. Examples include, but are not limited to, cyclopropenyl, cyclobutenyl, cyclopentenyl, or cyclohexenyl, and the like.

“Oxocycloalkenyl” means a monocyclic monovalent hydrocarbon radical of three to ten carbon atoms containing one or two double bond(s) and an oxo group, and is optionally substituted with one or two substituents independently selected from alkyl, halo, alkoxy, hydroxy, cyano, and cyanoalkyl. Examples include, but are not limited to, 3-oxocyclohex-1-enyl, and the like.

“Cyanoalkyl” means a linear monovalent hydrocarbon radical of one to six carbon atoms or a branched monovalent hydrocarbon radical of three to six carbons substituted with cyano e.g., cyanomethyl, cyanoethyl, and the like.

“Carboxy” means —COOH.

“Dialkylamino” means a —NRR′ radical where R and R′ are alkyl as defined above, e.g., dimethylamino, methylethylamino, and the like.

“Disubstituted amino” means a —NRR′ radical where R and R′ are independently alkyl, haloalkyl, hydroxyalkyl, alkoxyalkyl, or alkylcarbonyl, each as defined herein, e.g., dimethylamino, ethylmethylamino, bis-hydroxyethylamino, bis-methoxyethylamino, diethylaminoethylamino, and the like.

“Fused phenyl” means phenyl that is fused to cycloalkyl, cycloalkenyl, or heterocyclyl, each as defined herein. The fused phenyl can be attached to -L- of Formula (I) at any ring atom.

“Halo” means fluoro, chloro, bromo, or iodo, preferably fluoro or chloro.

“Haloalkyl” means alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C, unless stated otherwise. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group.

“Heterocyclylalkyl” or “heterocycloalkyl” means a -(alkylene)-R radical where R is heterocyclyl ring as defined above e.g., tetraydrofuranylmethyl, piperazinylmethyl, morpholinylethyl, and the like.

“Heterocyclylene” means a divalent heterocyclyl, as defined above, unless stated otherwise. When heterocyclylene contains 4, 5, or 6 rings atoms, it may be referred to herein as 4 to 6 membered heterocyclylene.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heteroarylene” means a divalent heteroaryl radical as defined above.

“Heteroaralkyl” means a -(alkylene)-R radical where R is heteroaryl as defined above, e.g., pyridinylmethyl, and the like. When the heteroaryl ring in heteroaralkyl contains 5- or 6 ring atoms it is also referred to herein as 5- or 6-membered heteroaralkyl.

The term “oxo,” as used herein, alone or in combination, refers to ═(O).

“Optionally substituted aryl” means aryl that is optionally substituted with one, two, or three substituents independently selected from alkyl, hydroxyl, cycloalkyl, carboxy, alkoxycarbonyl, hydroxy, alkoxy, alkylthio, alkylsulfonyl, amino, alkylamino, dialkylamino, halo, haloalkyl, haloalkoxy, and cyano.

“Optionally substituted heteroaryl” means heteroaryl as defined above that is optionally substituted with one, two, or three substituents independently selected from alkyl, alkylthio, alkylsulfonyl, hydroxyl, cycloalkyl, carboxy, alkoxycarbonyl, hydroxy, alkoxy, halo, haloalkyl, haloalkoxy, amino, alkylamino, dialkylamino, and cyano.

“Optionally substituted heterocyclyl” means heterocyclyl as defined above that is optionally substituted with one, two, or three substituents independently selected from alkyl, alkylthio, alkylsulfonyl, hydroxyl, cycloalkyl, carboxy, alkoxycarbonyl, hydroxy, hydroxyalkyl, alkoxy, alkoxyalkyl, aminoalkyl, halo, haloalkyl, haloalkoxy, and cyano, unless stated otherwise.

“Optionally substituted heterocyclylene” is divalent optionally substituted heterocyclyl as defined above.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

“Spirocycloalkyl” means a saturated bicyclic ring having 6 to 10 ring carbon atoms wherein the rings are connected through only one atom, the connecting atom is also called the spiroatom, most often a quaternary carbon (“spiro carbon”). The spirocycloalkyl ring is optionally substituted with one or two substituents independently selected from alkyl, halo, alkoxy, hydroxy, and cyano. Representative examples include, but are not limited to, spiro[3.3]heptane, spiro[3.4]octane, spiro[3.5]nonane, spiro[4.4]nonane (1:2:1:1), and the like.

“Spiroheterocyclyl” means a saturated bicyclic ring having 6 to 10 ring atoms in which one, two, or three ring atoms are heteroatom selected from N, O, or S(O)ₙ, where n is an integer from 0 to 2, the remaining ring atoms being C and the rings are connected through only one atom, the connecting atom is also called the spiroatom, most often a quaternary carbon (“spiro carbon”). The spiroheterocyclyl ring is optionally substituted with one, two, or three substituents independently selected from alkyl, alkylthio, alkylsulfonyl, hydroxyl, cycloalkyl, carboxy, alkoxycarbonyl, hydroxy, hydroxyalkyl, alkoxy, alkoxyalkyl, aminoalkyl, halo, haloalkyl, haloalkoxy, and cyano. Representative examples include, but are not limited to, 2,6-diazaspiro[3.3]heptane, 2,6-diazaspiro[3.4]octane, 2-azaspiro[3.4]octane, 2-azaspiro[3.5]nonane, 2,7-diazaspiro[4.4]nonane, and the like.

“Substantially identical” as used herein refers to measured physical characteristics that are comparable in value or data traces that are comparable in peak position and amplitude or intensity within the scope of variations that are typically associated with sample positioning or handling or the identity of the instrument employed to acquire the traces or physical characteristics or due to other variations or fluctuations normally encountered within or between laboratory environments or analytical instrumentation.

When needed, any definition herein may be used in combination with any other definition to describe a composite structural group. By convention, the trailing element of any such definition is that which attaches to the parent moiety. For example, the composite group alkoxyalkyl means that an alkoxy group attached to the parent molecule through an alkyl group.

The present disclosure also includes protected derivatives of compounds of Formula (I).

For example, when compounds of Formula (I) contain groups such as hydroxy, carboxy, thiol or any group containing a nitrogen atom(s), these groups can be protected with suitable protecting groups. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes polymorphic forms of compounds of Formula (I) or a pharmaceutically acceptable salt thereof. Polymorphs are different crystalline forms of a compound that differ in arrangements of the molecules of that compound in a crystal lattice. Therefore, a single compound may give rise to a variety of polymorphic forms. The polymorphs of a compound usually have different melting points, solubilities, densities and optical properties. Polymorphic forms of a compound can be distinguished by several techniques such as X-ray diffractometry, IR or Raman spectroscopy.

The term “prodrug” refers to a compound that is made more active in vivo. Certain compounds of Formula (I) may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds of Formula (I) are structurally modified forms of the compound that readily undergo chemical changes under physiological conditions to provide the active compound. Prodrugs are often useful because, in some situations, they may be easier to administer than the compound, or parent drug. They may, for instance, be bioavailable by oral administration whereas the parent drug is not. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound of Formula (I).

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, PA, 1985, which is incorporated herein by reference in its entirety.

The compounds of Formula (I) may have asymmetric centers. Compounds of Formula (I) containing an asymmetrically substituted atom may be isolated in optically active or racemic forms. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. All chiral, diastereomeric, all mixtures of chiral or diastereomeric forms, and racemic forms are within the scope of this disclosure, unless the specific stereochemistry or isomeric form is specifically indicated.

Certain compounds of Formula (I) can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all hydrates of a compound of Formula (I) are within the scope of this disclosure.

The compounds of Formula (I) may also contain unnatural amounts of isotopes at one or more of the atoms that constitute such compounds. Unnatural amounts of an isotope may be defined as ranging from the amount found in nature to an amount 100% of the atom in question. that differ only in the presence of one or more isotopically enriched atoms. Exemplary isotopes that can be incorporated into a compound of Formula (I) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds disclosed herein, including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

Certain structures provided herein are drawn with one or more floating substituents. Unless provided otherwise or otherwise clear from the context, the substituent(s) may be present on any atom of the ring to which it is attached, where chemically feasible and valency rules permitting. For example, in the structure:

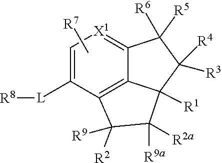

the R⁷ substituent can replace any hydrogen on the benzo portion of the tricyclic ring, including the hydrogen of CH when X¹ is CH.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “tumor,” as used herein, refers to all neoplastic cell growth and proliferation, whether malignant or benign, and all pre-cancerous and cancerous cells and tissues.

The terms “cancer,” “cancerous,” and “tumor” are not mutually exclusive as referred to herein.

The present disclosure includes a polymorph form of Compound 1. Polymorphs are different crystalline forms of a compound that differ in arrangements of the molecules of that compound in a crystal lattice. Therefore, a single compound may give rise to a variety of polymorph forms. The polymorphs of a compound usually have different melting points, solubilities, densities and optical properties. Polymorph forms of a compound can be distinguished by a number of techniques well known in the art such as X-ray diffractometry, IR or Raman spectroscopy.

“XRPD” means X-ray powder diffraction, an analytical technique which measures the diffraction of X-rays in the presence of a solid component. Materials which are crystalline and have regular repeating arrays of atoms generate a distinctive powder pattern.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The phrase “heteroaryl wherein the heteroaryl is optionally substituted with R^{d}, R^{e}, and R^{f} independently selected from hydrogen, alkyl, haloalkyl, haloalkoxy, alkoxy, hydroxy, halo, and cyano” in the definition of R⁹ in Formula (I) (and similar phrases used to define other groups in Formula (I)) is intended to cover heteroaryl that is unsubstituted and heteroaryl that is substituted with any one of R^{d}, R^{e}, and R^{f}.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” or “administering in combination with” means the administration of two or more therapeutic agents to treat a disease or disorder described in the present disclosure. Such administration encompasses co-administration of these therapeutic agents in a simultaneous manner, such as in a single capsule or tablet having a fixed ratio of active ingredients or in multiple, separate capsules or tablets for each active ingredient. In addition, such administration also encompasses use of each type of therapeutic agent in a sequential manner. In either case, the treatment regimen will provide beneficial effects of the drug combination in treating the conditions or disorders described herein.

“The term “chemotherapy” and “anti-cancer agent” are used interchangeably herein.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “synergy” or “synergistic” are used to mean that the result of the combination of a HIF-2α inhibitor or a pharmaceutically acceptable salt thereof and one or more anti-cancer agent is greater than the sum of each compound individually. This improvement in the disease, condition or disorder being treated is a “synergistic” effect.

A “synergistic amount” is an amount of the combination of a HIF-2α inhibitor or a pharmaceutically acceptable salt thereof and one or more anti-cancer agent that results in a synergistic effect, as “synergistic” is defined herein.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting (stabilizing) or reducing the development of the disease or its clinical symptoms; or
(3) relieving the disease, i.e., causing regression of the disease or its clinical symptoms.

A “therapeutically effective amount” means the amount of a compound of the present disclosure or a pharmaceutically acceptable salt thereof that, when administered to a patient for treating a disease, is sufficient to affect such treatment for the disease. The “therapeutically effective amount” will vary depending on the compound, the disease and its severity and the age, weight, etc., of the mammal to be treated.

The terms “inhibiting” and “reducing,” or any variation of these terms in relation of HIF-2a, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of HIF-2α activity compared to normal.

Representative HIF-2α inhibitors of Formula (I) are disclosed in Compound Table I below:

TABLE I

| Compound | | |
|---|---|---|
| # | Structure | Name |
| 1 | 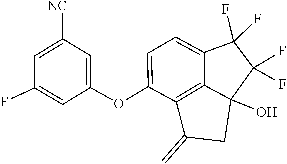 [SMILES: C=C1CC2(O)c3c(ccc(Oc4cc(F)cc(C#N)c4)c31)C(F)(F)C2(F)F] | 3-fluoro-5-((3,3,4,4-tetrafluoro-2a-hydroxy-1- methylene-2,2a,3,4-tetrahydro-1H- cyclopenta[cd]inden-7-yl)oxy)benzonitrile |
| 2 | 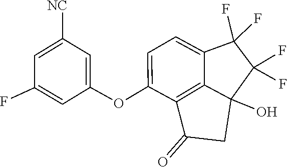 [SMILES: N#Cc1cc(F)cc(Oc2ccc3c4c2C(=O)CC4(O)C(F)(F)C3(F)F)c1] | 3-fluoro-5-((3,3,4,4-tetrafluoro-2a-hydroxy-1-oxo- 2,2a,3,4-tetrahydro-1H-cyclopenta[cd]inden-7- yl)oxy)benzonitrile |
| 3 | 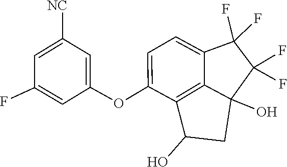 [SMILES: N#Cc1cc(F)cc(Oc2ccc3c4c2C(O)CC4(O)C(F)(F)C3(F)F)c1] | 3-fluoro-5-((3,3,4,4-tetrafluoro-1,2a-dihydroxy- 2,2a,3,4-tetrahydro-1H-cyclopenta[cd]inden-7- yl)oxy)benzonitrile |
| 4 | 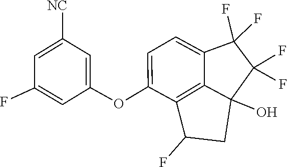 [SMILES: N#Cc1cc(F)cc(Oc2ccc3c4c2C(F)CC4(O)C(F)(F)C3(F)F)c1] | 3-fluoro-5-((1,3,3,4,4-pentafluoro-2a-hydroxy- 2,2a,3,4-tetrahydro-1H-cyclopenta[cd]inden-7- yl)oxy)benzonitrile |
| 5 | 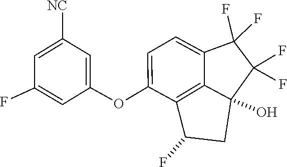 [SMILES: N#Cc1cc(F)cc(Oc2ccc3c4c2[C@@H](F)C[C@]4(O)C(F)(F)C3(F)F)c1] | 3-fluoro-5-(((1S,2aR)-1,3,3,4,4-pentafluoro-2a- hydroxy-2,2a,3,4-tetrahydro-1H- cyclopenta[cd]inden-7-yl)oxy)benzonitrile |
| 6 | 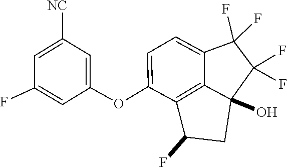 [SMILES: N#Cc1cc(F)cc(Oc2ccc3c4c2[C@H](F)C[C@@]4(O)C(F)(F)C3(F)F)c1] | 3-fluoro-5-(((1R,2aS)-1,3,3,4,4-pentafluoro-2a- hydroxy-2,2a,3,4-tetrahydro-1H- cyclopenta[cd]inden-7-yl)oxy)benzonitrile |
| 7 | 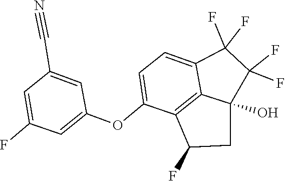 [SMILES: N#Cc1cc(F)cc(Oc2ccc3c4c2[C@H](F)C[C@]4(O)C(F)(F)C3(F)F)c1] | 3-fluoro-5-(((1R,2aR)-1,3,3,4,4-pentafluoro-2a- hydroxy-2,2a,3,4-tetrahydro-1H- cyclopenta[cd]inden-7-yl)oxy)benzonitrile |
| 8 | 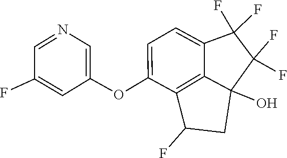 [SMILES: OC12CC(F)c3c(Oc4cncc(F)c4)ccc(c31)C(F)(F)C2(F)F] | 1,3,3,4,4-pentafluoro-7-((5-fluoropyridin-3- yl)oxy)-1,2,3,4-tetrahydro-2aH- cyclopenta[cd]inden-2a-ol |
| 9 | 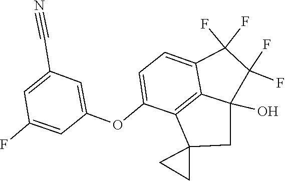 [SMILES: N#Cc1cc(F)cc(Oc2ccc3c4c2C2(CC2)CC4(O)C(F)(F)C3(F)F)c1] | 3-fluoro-5-((3,3,4,4-tetrafluoro-2a-hydroxy- 2,2a,3,4-tetrahydrospiro[cyclopenta[cd]indene- 1,1′-cyclopropan]-7-yl)oxy)benzonitrile |
| 10 | 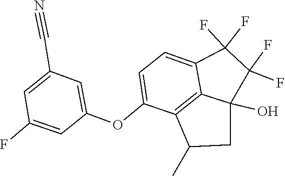 [SMILES: CC1CC2(O)c3c(ccc(Oc4cc(F)cc(C#N)c4)c31)C(F)(F)C2(F)F] | 3-fluoro-5-((3,3,4,4-tetrafluoro-2a-hydroxy-1- methyl-2,2a,3,4-tetrahydro-1H- cyclopenta[cd]inden-7-yl)oxy)benzonitrile |
| 11 | 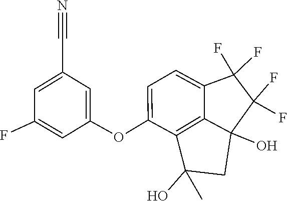 [SMILES: CC1(O)CC2(O)c3c(ccc(Oc4cc(F)cc(C#N)c4)c31)C(F)(F)C2(F)F] | 3-fluoro-5-((3,3,4,4-tetrafluoro-1,2a-dihydroxy-1- methyl-2,2a,3,4-tetrahydro-1H- cyclopenta[cd]inden-7-yl)oxy)benzonitrile |
| 12 | 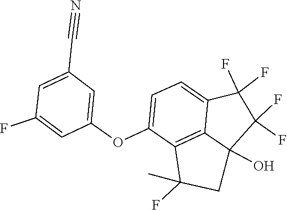 [SMILES: CC1(F)CC2(O)c3c(ccc(Oc4cc(F)cc(C#N)c4)c31)C(F)(F)C2(F)F] | 3-fluoro-5-((1,3,3,4,4-pentafluoro-2a-hydroxy-1- methyl-2,2a,3,4-tetrahydro-1H- cyclopenta[cd]inden-7-yl)oxy)benzonitrile |
| 13 | 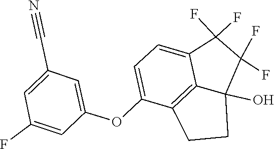 [SMILES: N#Cc1cc(F)cc(Oc2ccc3c4c2CCC4(O)C(F)(F)C3(F)F)c1] | 3-fluoro-5-((3,3,4,4-tetrafluoro-2a-hydroxy- 2,2a,3,4-tetrahydro-1H-cyclopenta[cd]inden-7- yl)oxy)benzonitrile |
| 14 | 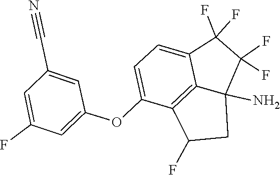 [SMILES: N#Cc1cc(F)cc(Oc2ccc3c4c2C(F)CC4(N)C(F)(F)C3(F)F)c1] | 3-((2a-amino-1,3,3,4,4-pentafluoro-2,2a,3,4- tetrahydro-1H-cyclopenta[cd]inden-7-yl)oxy)-5- fluorobenzonitrile |
| 15 | 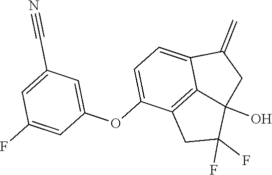 [SMILES: C=C1CC2(O)c3c1ccc(Oc1cc(F)cc(C#N)c1)c3CC2(F)F] | 3-((3,3-difluoro-2a-hydroxy-1-methylene-2,2a,3,4- tetrahydro-1H-cyclopenta[cd]inden-5-yl)oxy)-5- fluorobenzonitrile |
| 16 | 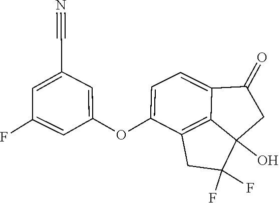 [SMILES: N#Cc1cc(F)cc(Oc2ccc3c4c2CC(F)(F)C4(O)CC3=O)c1] | 3-((3,3-difluoro-2a-hydroxy-1-oxo-2,2a,3,4- tetrahydro-1H-cyclopenta[cd]inden-5-yl)oxy)-5- fluorobenzonitrile |
| 17 | 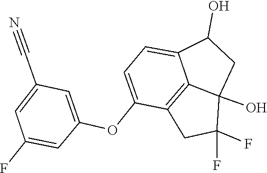 [SMILES: N#Cc1cc(F)cc(Oc2ccc3c4c2CC(F)(F)C4(O)CC3O)c1] | 3-((3,3-difluoro-1,2a-dihydroxy-2,2a,3,4- tetrahydro-1H-cyclopenta[cd]inden-5-yl)oxy)-5- fluorobenzonitrile |
| 18 | 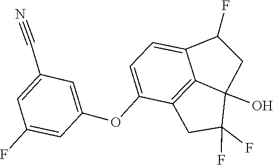 [SMILES: N#Cc1cc(F)cc(Oc2ccc3c4c2CC(F)(F)C4(O)CC3F)c1] | 3-fluoro-5-((1,3,3-trifluoro-2a-hydroxy-2,2a,3,4- tetrahydro-1H-cyclopenta[cd]inden-5- yl)oxy)benzonitrile |
| 19 | 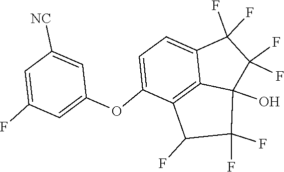 [SMILES: N#Cc1cc(F)cc(Oc2ccc3c4c2C(F)C(F)(F)C4(O)C(F)(F)C3(F)F)c1] | 3-fluoro-5-((1,2,2,3,3,4,4-heptafluoro-2a-hydroxy- 2,2a,3,4-tetrahydro-1H-cyclopenta[cd]inden-7- yl)oxy)benzonitrile |
| 20 | 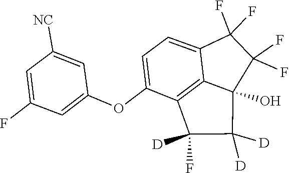 [SMILES: [2H]C1([2H])[C@@]2(O)c3c(ccc(Oc4cc(F)cc(C#N)c4)c3[C@@]1([2H])F)C(F)(F)C2(F)F] | 3-fluoro-5-(((1S,2aR)-1,3,3,4,4-pentafluoro-2a- hydroxy-2,2a,3,4-tetrahydro-1H- cyclopenta[cd]inden-7-yl-1,2,2- d3)oxy)benzonitrile |
| 21 | 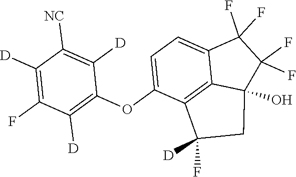 [SMILES: [2H]c1c(F)c([2H])c(Oc2ccc3c4c2[C@@]([2H])(F)C[C@]4(O)C(F)(F)C3(F)F)c([2H])c1C#N] | 3-fluoro-5-(((1S,2aR)-1,3,3,4,4-pentafluoro-2a- hydroxy-2,2a,3,4-tetrahydro-1H- cyclopenta[cd]inden-7-yl-1-d)oxy)benzonitrile- 2,4,6-d3 |
| 22 | 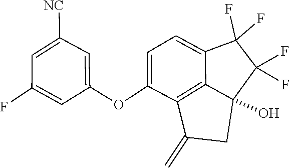 [SMILES: C=C1C[C@@]2(O)c3c(ccc(Oc4cc(F)cc(C#N)c4)c31)C(F)(F)C2(F)F] | (R)-3-fluoro-5-((3,3,4,4-tetrafluoro-2a-hydroxy-1- methylene-2,2a,3,4-tetrahydro-1H- cyclopenta[cd]inden-7-yl)oxy)benzonitrile |
| 23 | 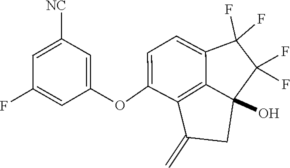 [SMILES: C=C1C[C@]2(O)c3c(ccc(Oc4cc(F)cc(C#N)c4)c31)C(F)(F)C2(F)F] | (S)-3-fluoro-5-((3,3,4,4-tetrafluoro-2a-hydroxy-1- methylene-2,2a,3,4-tetrahydro-1H- cyclopenta[cd]inden-7-yl)oxy)benzonitrile |
| 24 | 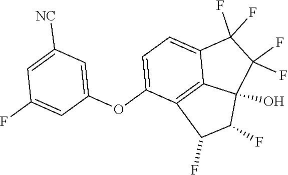 [SMILES: N#Cc1cc(F)cc(Oc2ccc3c4c2[C@@H](F)[C@@H](F)[C@]4(O)C(F)(F)C3(F)F)c1] | 3-fluoro-5-(((1R,2S,2aS)-1,2,3,3,4,4-hexafluoro- 2a-hydroxy-2,2a,3,4-tetrahydro-1H-cyclopenta[cd] inden-7-yl)oxy)benzonitrile |
| 25 | 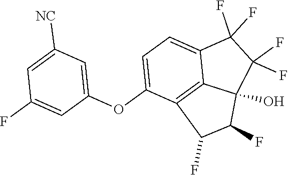 [SMILES: N#Cc1cc(F)cc(Oc2ccc3c4c2[C@@H](F)[C@H](F)[C@]4(O)C(F)(F)C3(F)F)c1] | 3-fluoro-5-(((1R,2R,2aS)-1,2,3,3,4,4-hexafluoro- 2a-hydroxy-2,2a,3,4-tetrahydro-1H-cyclopenta[cd] inden-7-yl)oxy)benzonitrile |

### EMBODIMENTS

In further embodiments 1-55 below, the present disclosure includes:

1. In embodiment 1, provided is a method of treating bladder cancer as described in the first aspect.

2. In embodiment 2, the method of embodiment 1 is wherein the HIF-2α inhibitor is a compound of Formula (I), or a pharmaceutically acceptable salt thereof.

2a. In embodiment 2a, the method of embodiment 1 or 2 is wherein the HIF-2α inhibitor is a compound of Formula (I), or a pharmaceutically acceptable salt thereof, wherein only R⁹ of R², R^{2a}, R⁹, and R^{9a} can be halo. In a subembodiment of embodiment 2a, R⁹ is halo.

2b. In embodiment 2b, the method of embodiment 1 or 2 is wherein the HIF-2α inhibitor is a compound of Formula (I), or a pharmaceutically acceptable salt thereof, wherein only R⁹ and R^{2a} of R², R^{2a}, R⁹, and R^{9a} can be halo. In a subembodiment of embodiment 2b, R⁹ is halo.

3. In embodiment 3, the method of embodiment 2, 2a, or 2b is wherein the compound of Formula (I) or a pharmaceutically acceptable salt thereof, is wherein R³ and R⁴ are independently halo.

4. In embodiment 4, the method of embodiment 2, 2a, or 2b is wherein the compound of Formula (I) or a pharmaceutically acceptable salt thereof, is wherein R³ is halo and R⁴ is hydrogen.

5. In embodiment 5, the method of embodiment 2, 2a, 2b, 3, or 4 is wherein the compound of Formula (I) or a pharmaceutically acceptable salt thereof, is wherein R¹ is hydroxy.

6. In embodiment 6, the method of any one of embodiments 2 to 4 is wherein the compound of Formula (I) or a pharmaceutically acceptable salt thereof, is wherein R¹ is amino.

7. In embodiment 7, the method of any one of embodiments 2 to 6 is wherein the compound of Formula (I) or a pharmaceutically acceptable salt thereof, is wherein R⁶ is halo.

8. In embodiment 8, the method of any one of embodiments 2 to 6 is wherein the compound of Formula (I) or a pharmaceutically acceptable salt thereof, is wherein R⁶ is alkyl, preferably R⁶ is methyl.

9. In embodiment 9, the method of any one of embodiments 2 to 6 is wherein the compound of Formula (I) or a pharmaceutically acceptable salt thereof, is wherein R⁶ is hydrogen.

10. In embodiment 10, the method of any one of embodiments 3 to 6 is wherein the compound of Formula (I) or a pharmaceutically acceptable salt thereof, is wherein R⁶ is cycloalkyl, preferably cyclopropyl, cyclobutyl, cyclopentyl or cyclohexyl.

11. In embodiment 11, the method of any one of embodiments 2 to 10 is wherein the compound of Formula (I) or a pharmaceutically acceptable salt thereof, is wherein R⁵ is halo, preferably fluoro.

12. In embodiment 12, the method of any one of embodiments 2 to 10 is wherein the compound of Formula (I) or a pharmaceutically acceptable salt thereof, is wherein R⁵ is haloalkyl, preferably R⁵ is difluoromethyl or trifluoromethyl.

13. In embodiment 13, the method of any one of embodiments 2 to 10 is wherein the compound of Formula (I) or a pharmaceutically acceptable salt thereof, is wherein R⁵ is alkyl, preferably R⁵ is methyl or ethyl.

14. In embodiment 14, the method of any one of embodiments 2 to 10 is wherein the compound of Formula (I) or a pharmaceutically acceptable salt thereof, is wherein R⁵ is hydrogen or alkoxy.

15. In embodiment 15, the method of any one of embodiments 2 to 6 is wherein the compound of Formula (I) or a pharmaceutically acceptable salt thereof, is wherein R⁵ and R⁶ together with the carbon to which they are attached form 3 to 6 membered cycloalkylene, preferably cyclopropylene, cyclobutylene or cyclopentylene, each of which is optionally substituted with one or two fluoro.

16. In embodiment 16, the method of any one of embodiments 2 to 15 is wherein the compound of Formula (I) or a pharmaceutically acceptable salt thereof, is wherein X¹ is CR⁷.

17. In embodiment 17, the method of embodiment 2, 2a, or 2b is wherein the compound of Formula (I) or a pharmaceutically acceptable salt thereof, has the structure of formula (IIa1) or (IIb1):

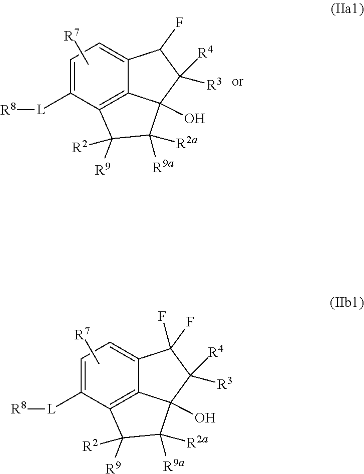

18. In embodiment 18, the method of embodiment 2, 2a, or 2b is wherein the compound of Formula (I) or a pharmaceutically acceptable salt thereof, has the structure of formula (IIa1′) or (IIb1′):

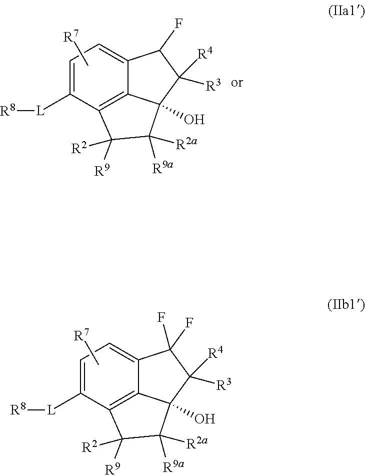

19. In embodiment 19, the method of embodiment 2, 2a, or 2b is wherein the compound of Formula (I) or a pharmaceutically acceptable salt thereof, has the structure of formula (IIa) or (IIb):

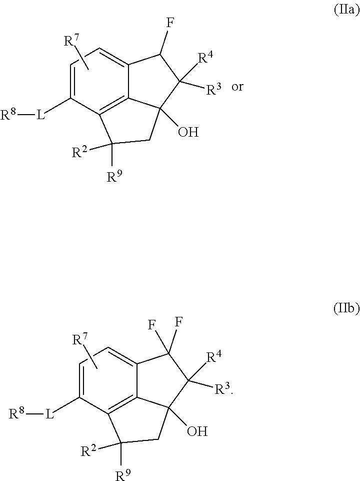

20. In embodiment 20, the method of embodiment 2, 2a, 2b, or 19 is wherein the compound of Formula (I) or a pharmaceutically acceptable salt thereof, has the structure of formula (IIa′) or (IIb′):

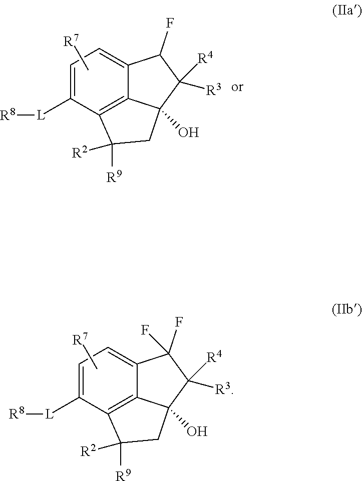

21. In embodiment 21, the method of embodiment 2, 2a, or 2b is wherein the compound of Formula (I) or a pharmaceutically acceptable salt thereof, has the structure of formula (IVa):

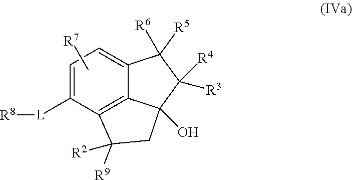

where R⁵ and R⁶ together with the carbon to which they are attached form a 3 to 6 membered cycloalkylene, preferably cyclopropylene, cyclobutylene or cyclopentylene, each of which is optionally substituted with one or two fluoro.

22. In embodiment 22, the method of any one of embodiments 17 to 21 is wherein the compound of Formulae (IIa1), (IIb1), (IIa1′), (IIb1′), (IIa), (IIb), (IIa′), (IIb′), and (IVa) or a pharmaceutically acceptable salt thereof, is wherein R³ is fluoro.

23. In embodiment 23, the method of any one of embodiments 17 to 21 is wherein the compound of Formulae (IIa1), (IIb1), (IIa1′), (IIb1′), (IIa), (IIb), (IIa′), (IIb′), and (IVa) or a pharmaceutically acceptable salt thereof is where R³ and R⁴ are fluoro.

24. In embodiment 24, the method of any one of embodiments 2 to 23 is wherein the compound of Formulae (I), (IIa1), (IIb1), (IIa1′), (IIb1′), (IIa), (IIb), (IIa′), (IIb′), and (IVa) or a pharmaceutically acceptable salt thereof, is wherein L is O, S, SO, SO₂, or NH.

25. In embodiment 25, the method of embodiment 24 is wherein the compound of Formulae (I), (IIa1), (IIb1), (IIa1′), (IIb1′), (IIa), (IIb), (IIa′), (IIb′), and (IVa) or a pharmaceutically acceptable salt thereof, is wherein L is O.

25a. In embodiment 25a, the method of embodiment 24 is wherein the compound of Formulae (I), (IIa1), (IIb1), (IIa1′), (IIb1′), (IIa), (IIb), (IIa′), (IIb′), and (IVa) or a pharmaceutically acceptable salt thereof, is wherein L is a bond and R⁸ is fused phenyl substituted as defined in the first aspect of the summary.

26. In embodiment 26, the method of any one of embodiments 2 to 25, and subembodiments contained therein, is wherein the compound of Formulae (I), (IIa1), (IIb1), (IIa1′), (IIb1′), (IIa), (IIb), (IIa′), (IIb′), and (IVa) or a pharmaceutically acceptable salt thereof, is wherein R⁸ is phenyl substituted with R^{a}, R^{b}, R^{c}, R^{g} and R^{h} wherein R^{a}, R^{b}, and R^{c} are independently selected from hydrogen, deuterium, alkyl, haloalkyl, haloalkyloxy, alkoxy, hydroxy, halo, cyano, hydroxyalkyl, alkoxyalkyl, aminoalkyl, optionally substituted aryl, optionally substituted heteroaryl, and optionally substituted heterocyclyl and R^{g} and R^{h} are independently selected from hydrogen, deuterium, and halo.

27. In embodiment 28, the method of embodiment 26 and subembodiments contained therein, is wherein the compound of Formulae (I), (IIa1), (IIb1), (IIa1′), (IIb1′), (IIa), (IIb), (IIa′), (IIb′) and (IVa), or a pharmaceutically acceptable salt thereof, is wherein R⁸ is 3-chloro-5-fluorophenyl, 3,5-difluorophenyl, 3-fluoro-5-methoxyphenyl, 3-cyano-5-fluorophenyl, 3-chloro-5-cyanophenyl, 3-cyano-5-methylphenyl, 3-chloro-4-fluorophenyl, 3-chloro-5-fluorophenyl, 3-fluoro-5-methyphenyl, 3-cyanophenyl, 3-trifluoromethylphenyl, 3,4-dichlorophenyl, 3-chloro-2-methylphenyl, 3,5-dichlorophenyl, 3,5-dimethylphenyl, 2-chloro-6-methylphenyl, 2,6-difluorophenyl, 3,4,5-trifluorophenyl, 3,4-difluorophenyl, 4-fluoro-3-methylphenyl, 3-cyano-4-fluorophenyl, 3-cyano-5-difluoromethylphenyl or 3-cyano-5-fluoro-2,4,6-trideuteriophenyl. In a first subembodiment of embodiment 27, R¹⁰ is 3-cyano-5-fluorophenyl or 3-cyano-5-fluoro-2,4,6-trideuteriophenyl.

28. In embodiment 28, the method of any one of embodiments 2 to 25 and any subembodiments contained therein, is wherein the compound of Formulae (I), (IIa1), (IIb1), (IIa1′), (IIb1′), (IIa), (IIb), (IIa′), (IIb′), and (IVa) or a pharmaceutically acceptable salt thereof, is wherein R⁸ is cycloalkyl or cycloalkylalkyl each optionally substituted with one or two substituents independently selected from alkyl, halo, alkoxy, cyano, and hydroxy.

29. In embodiment 29, the method of any one of embodiments 2 to 25 and any subembodiments contained therein is wherein the compound of Formulae (I), (IIa1), (IIb1), (IIa1′), (IIb1′), (IIa), (IIb), (IIa′), (IIb′), and (IVa) or a pharmaceutically acceptable salt thereof, is wherein R⁸ is heteroaryl substituted with R^{a}, R^{b}, and R^{c} independently selected from hydrogen, alkyl, haloalkyl, haloalkyloxy, alkoxy, hydroxy, halo, cyano, hydroxyalkyl, alkoxyalkyl, aminoalkyl, optionally substituted aryl, optionally substituted heteroaryl, and optionally substituted heterocyclyl.

30. In embodiment 30, the method of any one of embodiments 2 to 25 is wherein the compound of Formulae (I), (IIa1), (IIb1), (IIa1′), (IIb1′), (IIa), (IIb), (IIa′), (IIb′) and (IVa) or a pharmaceutically acceptable salt, thereof, is wherein R⁸ is pyridin-3-yl, pyridin-2-yl, pyridazin-3-yl, pyridazin-4-yl, pyrimidin-5-yl, pyrimidin-2-yl, thien-2-yl, furan-2-yl, thiazol-5-yl, oxazol-5-yl, imidazol-5-yl, furan-3-yl, thien-3-yl, thiazol-4-yl, pyridin-4-yl, oxazol-2-yl, imidazol-2-yl, pyridin-2-yl, pyrazin-2-yl, or thiazol-2-yl, and R⁸ is substituted with R^{a}, R^{b}, and R^{c} wherein R^{a} and R^{b} are independently selected from hydrogen, methyl, methoxy, hydroxy, chloro, fluoro, difluoromethyl, trifluoromethyl, difluoromethoxy, and trifluoromethoxy and R^{c} is selected from hydrogen, methyl, cyano, chloro, fluoro, difluoromethyl, trifluoromethyl, difluoromethoxy, and trifluoromethoxy.

31. In embodiment 32, the method of any one of embodiments 2 to 30 is wherein the compound of Formulae (I), (IIa1), (IIb1), (IIa1′), (IIb1′), (IIa), (IIb), (IIa′), (IIb′) and (IVa) or a pharmaceutically acceptable salt thereof, is wherein R⁷ is hydrogen, methyl, ethyl, methoxy, fluoro, trifluoromethyl, or trifluoromethoxy, preferably R⁷ is hydrogen.

32. In embodiment 32, the method of any one of embodiments 2 to 31 is wherein the compound of Formulae (I), (IIa1), (IIb1), (IIa1′), (IIb1′), (IIa), (IIb), (IIa′), (IIb′) and (IVa) or a pharmaceutically acceptable salt thereof, is wherein R² is hydrogen, fluoro, methyl, or ethyl.

33. In embodiment 33, the method of any one of embodiments 2 to 32 is wherein the compound of Formulae (I), (IIa1), (IIb1), (IIa1′), (IIb1′), (IIa), (IIb), (IIa′), (IIB′) and (IVa) or a pharmaceutically acceptable salt thereof, is wherein R⁹ is hydrogen, alkyl, halo, hydroxy, or alkoxy.

34. In embodiment 34, the method of embodiment 33 is wherein the compound of Formulae (I), (IIa1), (IIb1), (IIa1′), (IIb1′), (IIa), (IIb), (IIa′), (IIb′) and (IVa) or a pharmaceutically acceptable salt thereof, is wherein R⁹ is hydrogen, methyl, hydroxy, or fluoro.

35. In embodiment 35, the method of embodiment 2 is wherein the compound of Formula (I) or a pharmaceutically acceptable salt thereof, has the structure of formula (VIIIa) or (VIIIb):

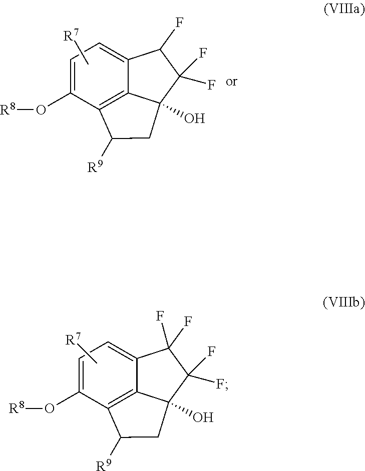

preferably the structure of formula (VIIIb).

36. In embodiment 36, the method of embodiment 1 is wherein the compound of Formula (I) or a pharmaceutically acceptable salt thereof, has the structure of formula (IXa) or (IXb):

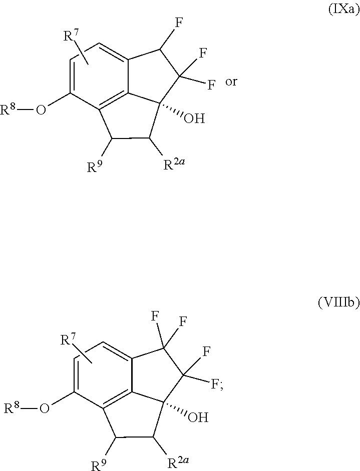

where R^{2a} is other than hydrogen.

37. In embodiment 37, the method of embodiment 35 or 36, is wherein R⁷ is hydrogen, R⁸ is 3-cyano-5-fluorophenyl, R⁹ and R^{2a} are halo.

38. In embodiment 38, the method of embodiment 2, is wherein the compound of Formula (I) is selected from:
3-fluoro-5-((3,3,4,4-tetrafluoro-2a-hydroxy-1-methylene-2,2a,3,4-tetrahydro-1H-cyclopenta[cd]inden-7-yl)oxy)benzonitrile;
3-fluoro-5-((3,3,4,4-tetrafluoro-2a-hydroxy-1-oxo-2,2a,3,4-tetrahydro-1H-cyclopenta[cd]inden-7-yl)oxy)benzonitrile;
3-fluoro-5-((3,3,4,4-tetrafluoro-1,2a-dihydroxy-2,2a,3,4-tetrahydro-1H-cyclopenta[cd]inden-7-yl)oxy)benzonitrile;
3-fluoro-5-((1,3,3,4,4-pentafluoro-2a-hydroxy-2,2a,3,4-tetrahydro-1H-cyclopenta[cd]inden-7-yl)oxy)benzonitrile;
3-fluoro-5-(((1S,2aR)-1,3,3,4,4-pentafluoro-2a-hydroxy-2,2a,3,4-tetrahydro-1H-cyclopenta[cd]inden-7-yl)oxy)benzonitrile;
3-fluoro-5-(((1R,2aS)-1,3,3,4,4-pentafluoro-2a-hydroxy-2,2a,3,4-tetrahydro-1H-cyclopenta[cd]inden-7-yl)oxy)benzonitrile;
3-fluoro-5-(((1R,2aR)-1,3,3,4,4-pentafluoro-2a-hydroxy-2,2a,3,4-tetrahydro-1H-cyclopenta[cd]inden-7-yl)oxy)benzonitrile;
1,3,3,4,4-pentafluoro-7-((5-fluoropyridin-3-yl)oxy)-1,2,3,4-tetrahydro-2aH-cyclopenta[cd]inden-2a-ol;
3-fluoro-5-((3,3,4,4-tetrafluoro-2a-hydroxy-2,2a,3,4-tetrahydrospiro[cyclopenta[cd]-indene-1,1′-cyclopropan]-7-yl)oxy)benzonitrile;
3-fluoro-5-((3,3,4,4-tetrafluoro-2a-hydroxy-1-methyl-2,2a,3,4-tetrahydro-1H-cyclopenta[cd]inden-7-yl)oxy)benzonitrile;
3-fluoro-5-((3,3,4,4-tetrafluoro-1,2a-dihydroxy-1-methyl-2,2a,3,4-tetrahydro-1H-cyclopenta[cd]inden-7-yl)oxy)benzonitrile;
3-fluoro-5-((1,3,3,4,4-pentafluoro-2a-hydroxy-1-methyl-2,2a,3,4-tetrahydro-1H-cyclopenta[cd]inden-7-yl)oxy)benzonitrile;
3-fluoro-5-((3,3,4,4-tetrafluoro-2a-hydroxy-2,2a,3,4-tetrahydro-1H-cyclopenta[cd]inden-7-yl)oxy)benzonitrile;
3-((2a-amino-1,3,3,4,4-pentafluoro-2,2a,3,4-tetrahydro-1H-cyclopenta[cd]inden-7-yl)oxy)-5-fluorobenzonitrile;
3-fluoro-5-((1,1,2a,3,3,4,4-heptafluoro-2,2a,3,4-tetrahydro-1H-cyclopenta[cd]inden-7-yl)oxy)benzonitrile;
3-((3,3-difluoro-2a-hydroxy-1-methylene-2,2a,3,4-tetrahydro-1H-cyclopenta[cd]inden-5-yl)oxy)-5-fluorobenzonitrile;
3-((3,3-difluoro-2a-hydroxy-1-oxo-2,2a,3,4-tetrahydro-1H-cyclopenta[cd]inden-5-yl)oxy)-5-fluorobenzonitrile;
3-((3,3-difluoro-1,2a-dihydroxy-2,2a,3,4-tetrahydro-1H-cyclopenta[cd]inden-5-yl)oxy)-5-fluorobenzonitrile;
3-fluoro-5-((1,3,3-trifluoro-2a-hydroxy-2,2a,3,4-tetrahydro-1H-cyclopenta[cd]inden-5-yl)oxy)benzonitrile;
3-fluoro-5-((1,2,2,3,3,4,4-heptafluoro-2a-hydroxy-2,2a,3,4-tetrahydro-1H-cyclopenta[cd]-inden-7-yl)oxy)benzonitrile;
3-fluoro-5-(((1S,2aR)-1,3,3,4,4-pentafluoro-2a-hydroxy-2,2a,3,4-tetrahydro-1H-cyclopenta[cd]inden-7-yl-1,2,2-d3)oxy)benzonitrile;
3-fluoro-5-(((1S,2aR)-1,3,3,4,4-pentafluoro-2a-hydroxy-2,2a,3,4-tetrahydro-1H-cyclopenta[cd]inden-7-yl-1-d)oxy)benzonitrile-2,4,6-d3;
(R)-3-fluoro-5-((3,3,4,4-tetrafluoro-2a-hydroxy-1-methylene-2,2a,3,4-tetrahydro-1H-cyclopenta[cd]inden-7-yl)oxy)benzonitrile;
(S)-3-fluoro-5-((3,3,4,4-tetrafluoro-2a-hydroxy-1-methylene-2,2a,3,4-tetrahydro-1H-cyclopenta[cd]inden-7-yl)oxy)benzonitrile;
3-fluoro-5-(((1R,2S,2aS)-1,2,3,3,4,4-hexafluoro-2a-hydroxy-2,2a,3,4-tetrahydro-1H-cyclopenta[cd] inden-7-yl)oxy)benzonitrile; and
3-fluoro-5-(((1R,2R,2aS)-1,2,3,3,4,4-hexafluoro-2a-hydroxy-2,2a,3,4-tetrahydro-1H-cyclopenta[cd] inden-7-yl)oxy)benzonitrile; or
   a pharmaceutically acceptable salt thereof.

39. In embodiment 39, the method of embodiment 1, is wherein the HIF-2α inhibitor is:
3-fluoro-5-((1,3,3,4,4-pentafluoro-2a-hydroxy-2,2a,3,4-tetrahydro-1H-cyclopenta-[cd]-inden-7-yl)oxy)benzonitrile;
3-(((1S,2S,3R)-2,3-difluoro-1-hydroxy-7-(methylsulfonyl)-2,3-dihydro-1H-inden-4-yl)-oxy)-5-fluorobenzonitrile;
   Novartis DFF332; or
   Arcus AB521; or
   a pharmaceutically acceptable salt thereof.

39a. In embodiment 39a, the method of embodiment 1, is wherein the HIF-2α inhibitor is:
3-(((1S,2S,3R)-2,3-difluoro-1-hydroxy-7-(methylsulfonyl)-2,3-dihydro-1H-inden-4-yl)-oxy)-5-fluorobenzonitrile;
(S)-1′-chloro-8-(difluoromethoxy)-8′,8′-difluoro-6-(trifluoromethyl)-7′,8′-dihydro-3H,6′H-spiro[imidazo[1,2-a]pyridine-2,5′-isoquinoline] (Novartis DFF332); or
   Arcus AB521; or
   a pharmaceutically acceptable salt thereof;
   wherein the bladder cancer is muscle-invasive bladder cancer.

40. In embodiment 40, the method of embodiment 1, 2, or 38, is wherein the HIF-2α inhibitor is 3-fluoro-5-((1,3,3,4,4-pentafluoro-2a-hydroxy-2,2a,3,4-tetrahydro-1H-cyclopenta[cd]inden-7-yl)oxy)-benzonitrile.

41. In embodiment 41, the method of embodiment 1, 2, or 40, is wherein the HIF-2α inhibitor is 3-fluoro-5-(((1S,2aR)-1,3,3,4,4-pentafluoro-2a-hydroxy-2,2a,3,4-tetrahydro-TH-cyclopenta[cd]inden-7-yl)oxy)-benzonitrile.

42. In embodiment 42, the method of embodiment 41, is wherein the 3-fluoro-5-(((1S,2aR)-1,3,3,4,4-pentafluoro-2a-hydroxy-2,2a,3,4-tetrahydro-TH-cyclopenta[cd]inden-7-yl)oxy)-benzonitrile is a crystalline solid, designated as Form A polymorph, having an X-ray powder diffraction pattern comprising peaks at angular positions 15.8 and 18.6, wherein the angular positions may vary by ±0.2020 as measured by X-ray powder diffraction using (Cu Ku) an X-ray wavelength of 1.5418 Å.

43. In embodiment 43, the method of embodiment 42, is wherein the Form A polymorph X-ray powder diffraction pattern further comprises peak at angular position 20.1, wherein the angular positions may vary by ±0.2° 2θ.

44. In embodiment 44, the method of embodiment 42, is wherein the Form A polymorph X-ray powder diffraction pattern further comprises peaks at angular positions 12.9 and 20.1, wherein the angular positions may vary by ±0.2° 2θ.

45. In embodiment 45, the method of embodiment 42, is wherein the Form A polymorph X-ray powder diffraction pattern further comprises peaks at angular positions 11.4, 12.9, and 20.1, wherein the angular positions may vary by ±0.2° 2θ.

46. In embodiment 46, the method of embodiment 42, is wherein the Form A polymorph X-ray powder diffraction pattern further comprises peaks at angular positions 10.1, 11.4, 12.9, and 20.1, wherein the angular positions may vary by ±0.2° 2θ.

47. In embodiment 47, the method of embodiment 42, is wherein the 3-fluoro-5-(((1S,2aR)-1,3,3,4,4-pentafluoro-2a-hydroxy-2,2a,3,4-tetrahydro-TH-cyclopenta[cd]inden-7-yl)oxy)-benzonitrile is a crystalline solid, designated as Form A polymorph, having an X-ray powder diffraction pattern comprising at least two, at least three, at least four, at least five, at least six peaks, at least seven peaks, or at least eight peaks at angular positions selected from Table 1 below, wherein the angular positions may vary by ±0.2° 2θ.

TABLE 1

| Angle | | |
|---|---|---|
| (2-Theta °) | d value (Å) | Intensity % |
| 9.2 | 9.59 | 6.9 |
| 10.1 | 8.79 | 19.5 |
| 11.4 | 7.75 | 21.6 |
| 12.9 | 6.87 | 21.6 |
| 13.7 | 6.48 | 15.2 |
| 14.0 | 6.33 | 3.5 |
| 15.4 | 5.75 | 3.8 |
| 15.8 | 5.59 | 100 |
| 16.6 | 5.35 | 9.8 |
| 17.4 | 5.09 | 6.7 |
| 18.0 | 4.93 | 10.2 |
| 18.6 | 4.77 | 25.3 |
| 19.6 | 4.53 | 15.3 |
| 20.1 | 4.41 | 24.3 |
| 20.7 | 4.29 | 4 |
| 21.0 | 4.23 | 3.9 |
| 21.4 | 4.15 | 13.2 |
| 21.7 | 4.09 | 18.5 |
| 22.1 | 4.03 | 10.6 |
| 22.6 | 3.94 | 5.3 |
| 23.8 | 3.74 | 3 |
| 24.1 | 3.69 | 4.5 |
| 25.0 | 3.55 | 14.7 |
| 26.0 | 3.43 | 13.9 |
| 26.7 | 3.33 | 3.4 |
| 27.2 | 3.28 | 6.4 |
| 27.6 | 3.23 | 4.4 |
| 27.8 | 3.20 | 3.8 |
| 28.2 | 3.16 | 7.3 |
| 29.0 | 3.07 | 6.2 |
| 29.6 | 3.02 | 9.3 |
| 30.1 | 2.97 | 4.4 |
| 30.6 | 2.92 | 3 |
| 31.0 | 2.88 | 2.9 |
| 31.7 | 2.82 | 3.5 |
| 31.9 | 2.80 | 4 |
| 32.4 | 2.76 | 5.1 |
| 33.6 | 2.66 | 5.4 |
| 34.2 | 2.62 | 3.3 |
| 34.6 | 2.59 | 4.3 |
| 35.4 | 2.53 | 3.2 |
| 35.8 | 2.50 | 3.3 |

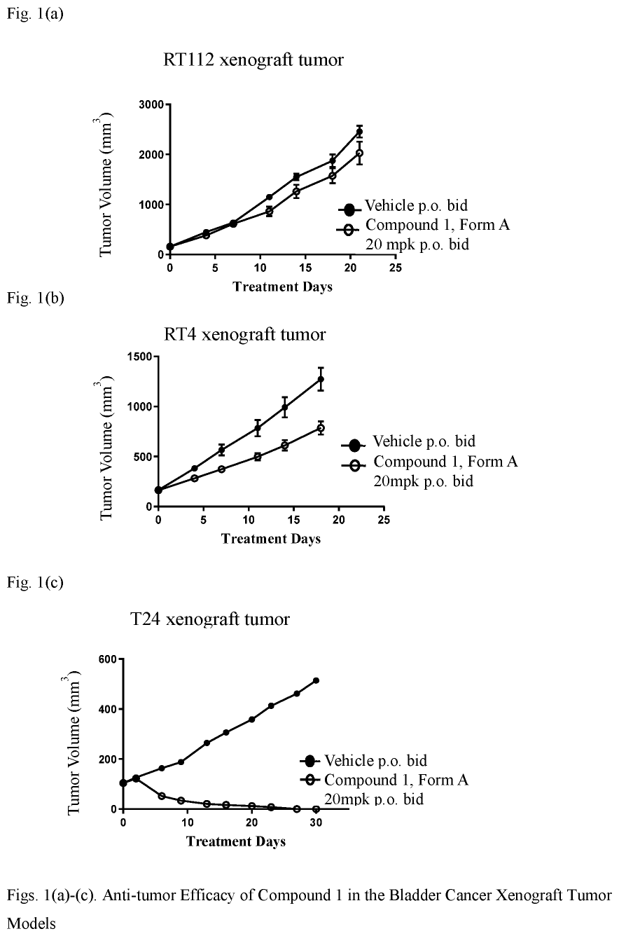

48. In embodiment 48, method of embodiment 47, is wherein the at least two, at least three, at least four, at least five, or at least six peaks are selected from 10.1, 11.4, 12.9, 13.7, 15.9, 18.0, 19.6, 20.1, 21.4, 21.7, 25.0, and 26.0, wherein the angular positions may vary by ±0.2° 2θ. 49. In embodiment 49, the method of embodiment 41, is wherein the 3-fluoro-5-(((1S,2aR)-1,3,3,4,4-pentafluoro-2a-hydroxy-2,2a,3,4-tetrahydro-1H-cyclopenta[cd]inden-7-yl)oxy)-benzonitrile is a crystalline solid, designated as Form A polymorph, having an X-ray powder diffraction pattern having an X-ray powder diffraction pattern substantially identical as shown in FIG. 1.

50. In embodiment 50, the method of any one of embodiments 42 to 48, is wherein the the angular positions of the X-ray powder diffraction pattern peaks denoted therein may vary by ±0.1° 2θ.

51. In embodiment 51, provided is a subembodiment of the third embodiment of the first aspect, where the HIF-2 inhibitor is selected from the group consisting of:

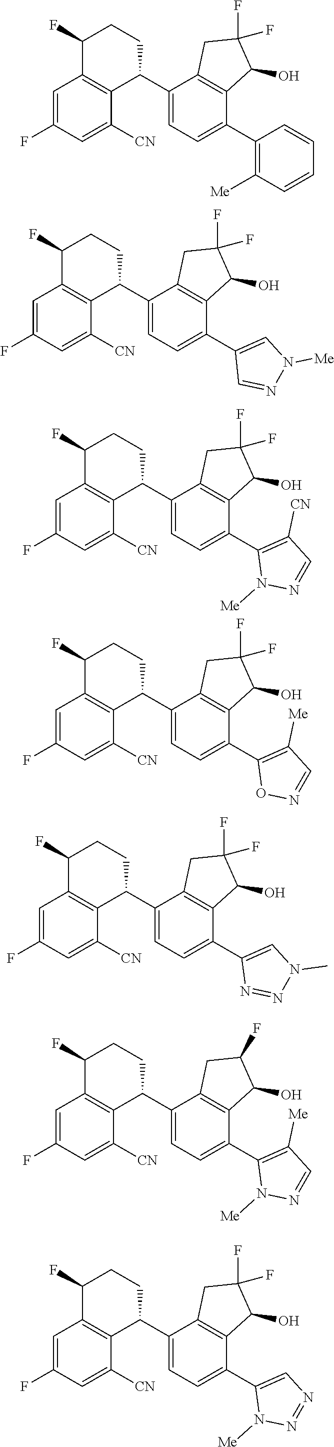

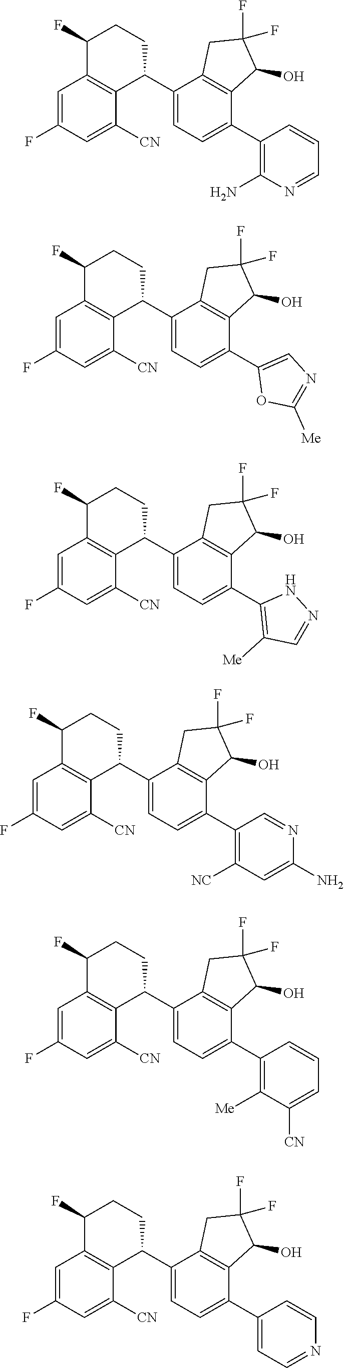

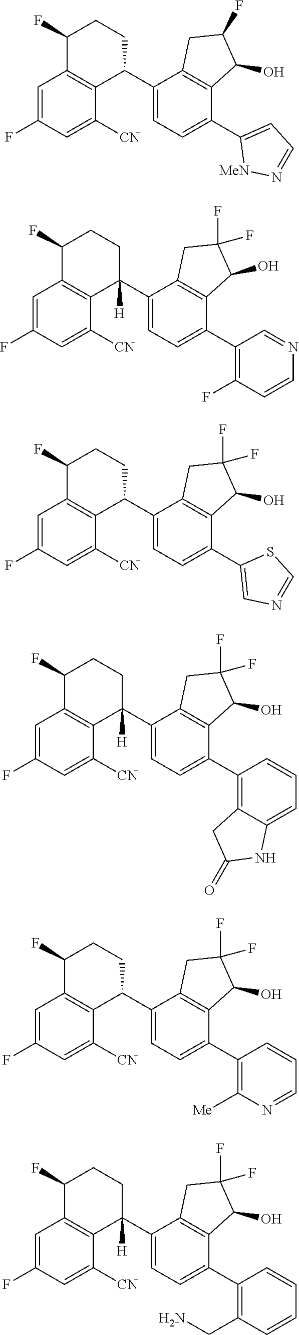

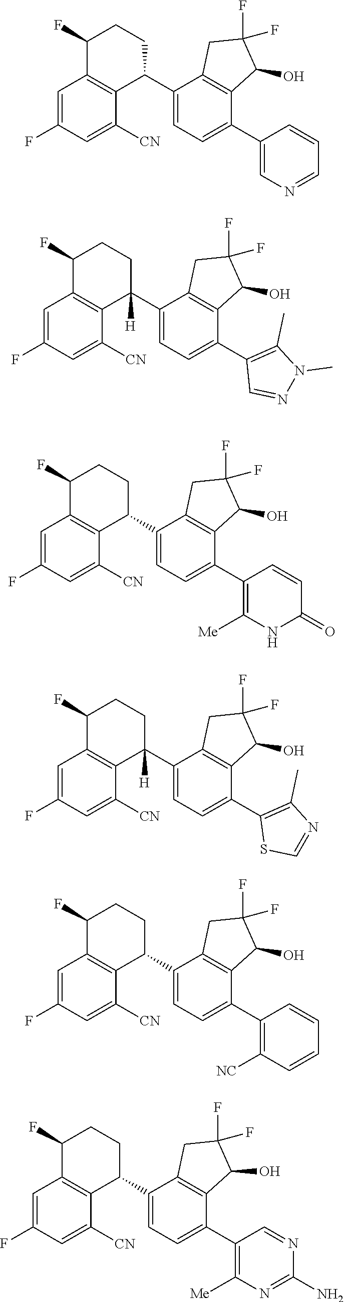

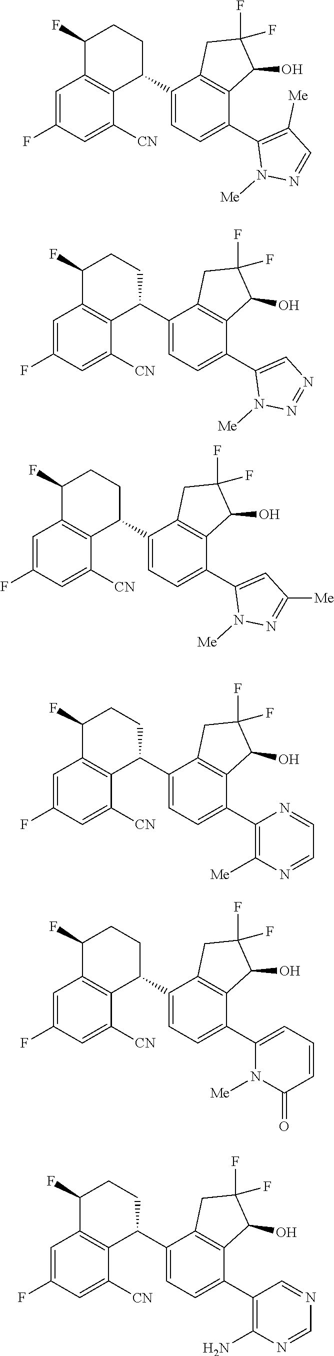

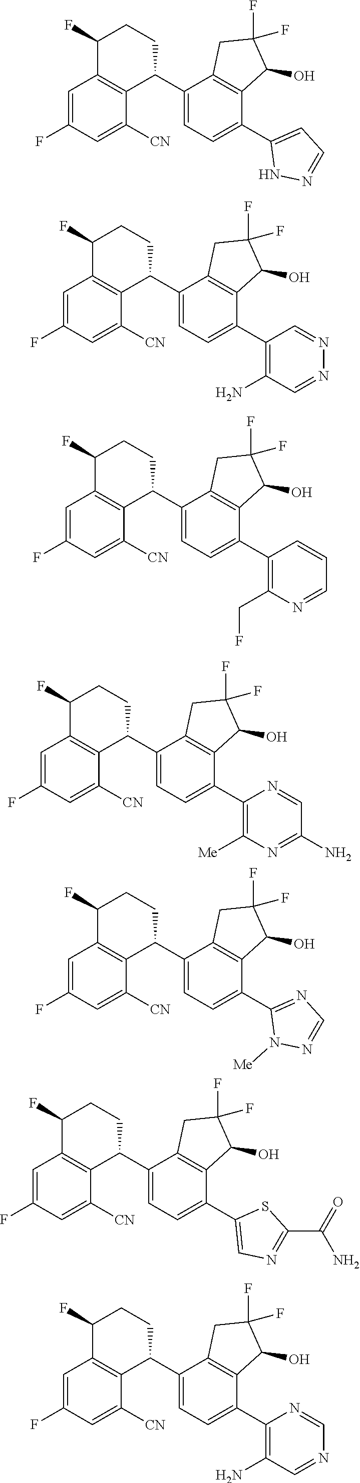

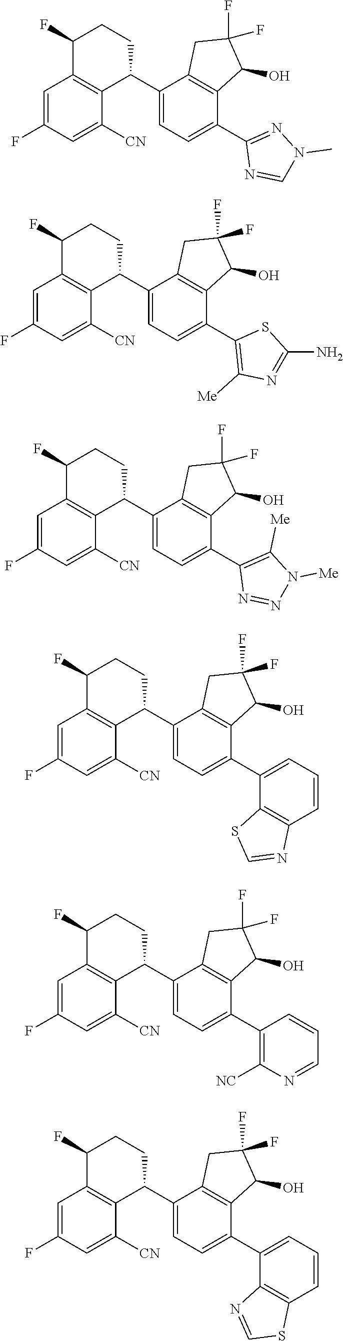

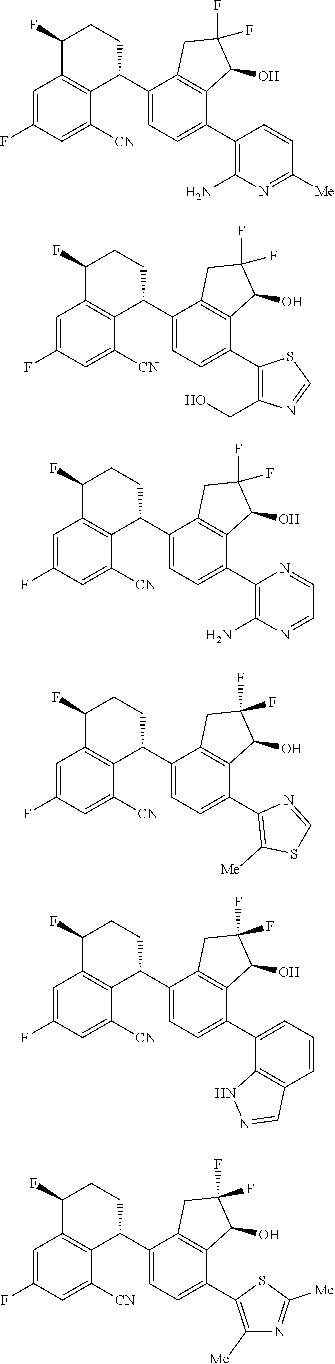

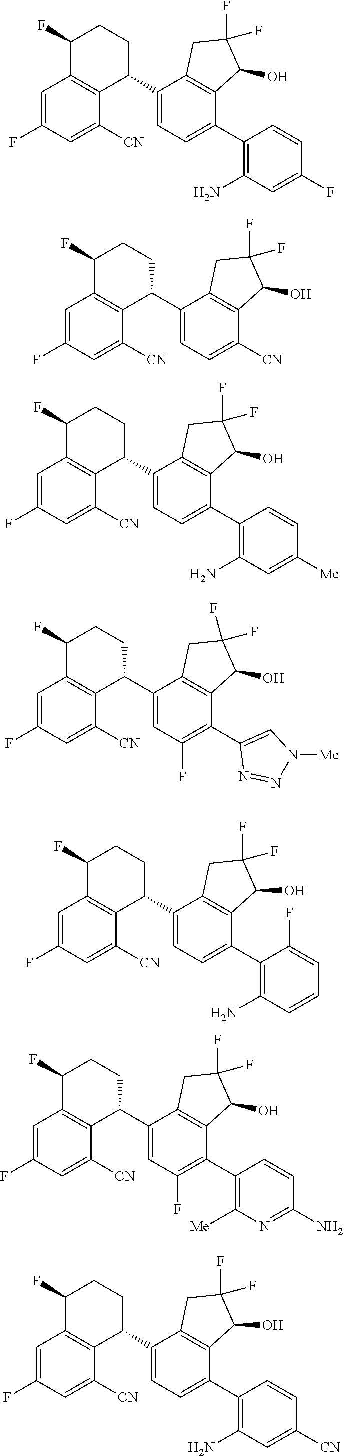

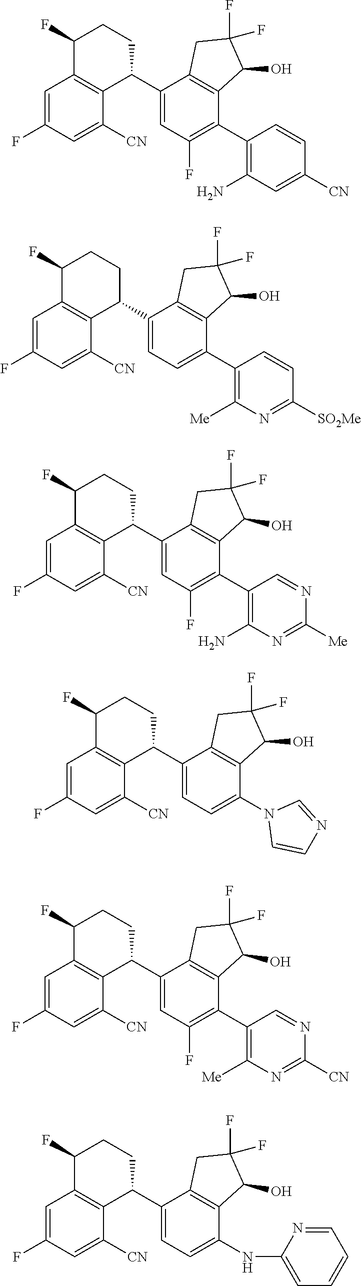

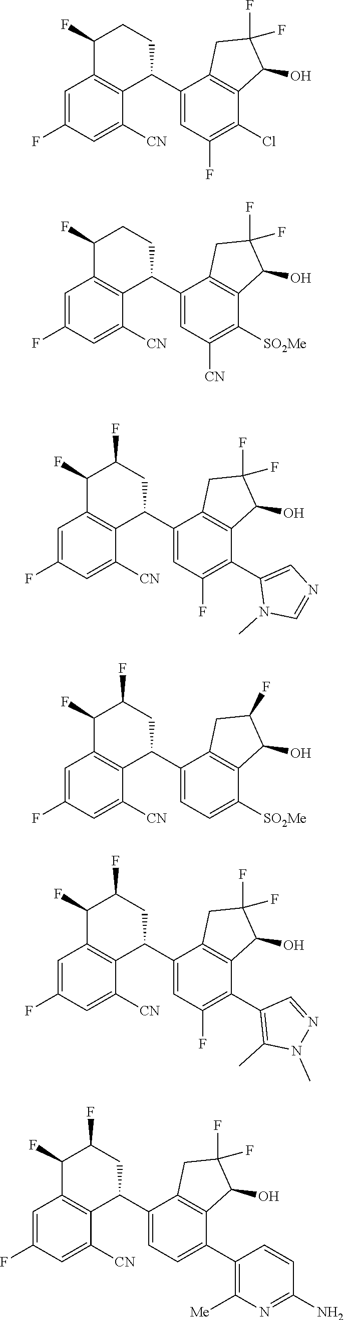 [SMILES: N#Cc1cc(F)cc2c1[C@@H](c1cc(F)c(Cl)c3c1CC(F)(F)[C@H]3O)CC[C@@H]2F ; CS(=O)(=O)c1c(C#N)cc([C@H]2CC[C@H](F)c3cc(F)cc(C#N)c32)c2c1[C@H](O)C(F)(F)C2 ; Cn1cncc1-c1c(F)cc([C@H]2C[C@H](F)[C@H](F)c3cc(F)cc(C#N)c32)c2c1[C@H](O)C(F)(F)C2 ; CS(=O)(=O)c1ccc([C@H]2C[C@H](F)[C@H](F)c3cc(F)cc(C#N)c32)c2c1[C@H](O)[C@H](F)C2 ; Cc1c(-c2c(F)cc([C@H]3C[C@H](F)[C@H](F)c4cc(F)cc(C#N)c43)c3c2[C@H](O)C(F)(F)C3)cnn1C ; Cc1nc(N)ccc1-c1ccc([C@H]2C[C@H](F)[C@H](F)c3cc(F)cc(C#N)c32)c2c1[C@H](O)C(F)(F)C2]

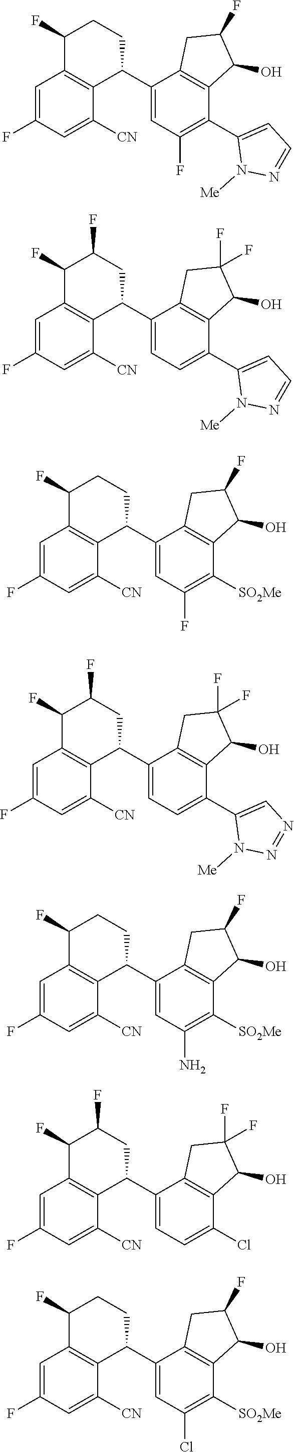

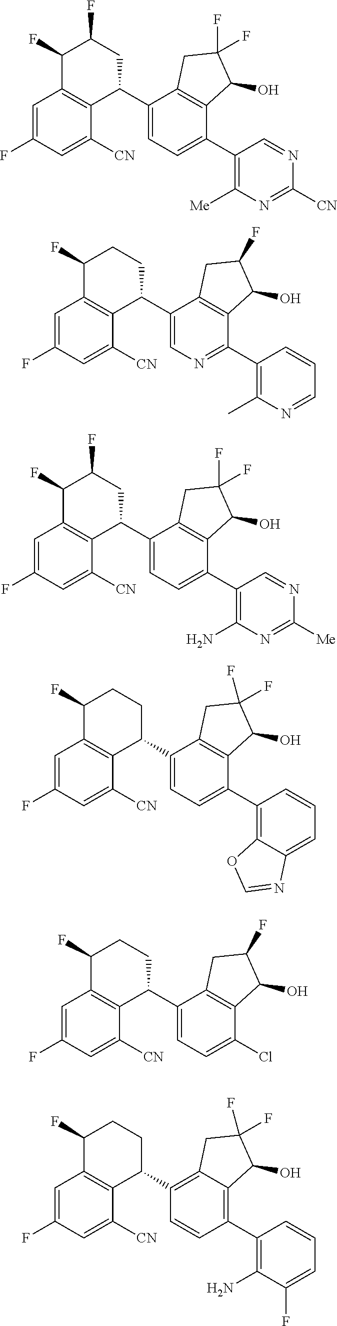

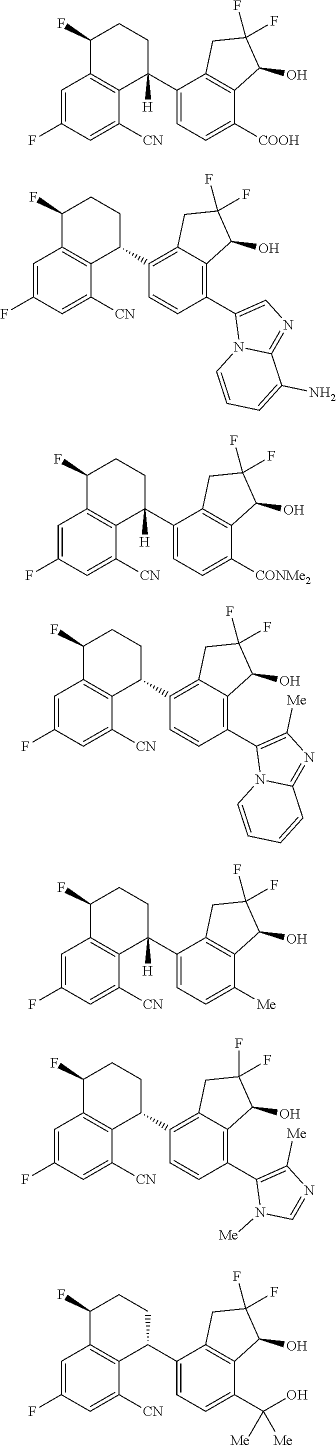

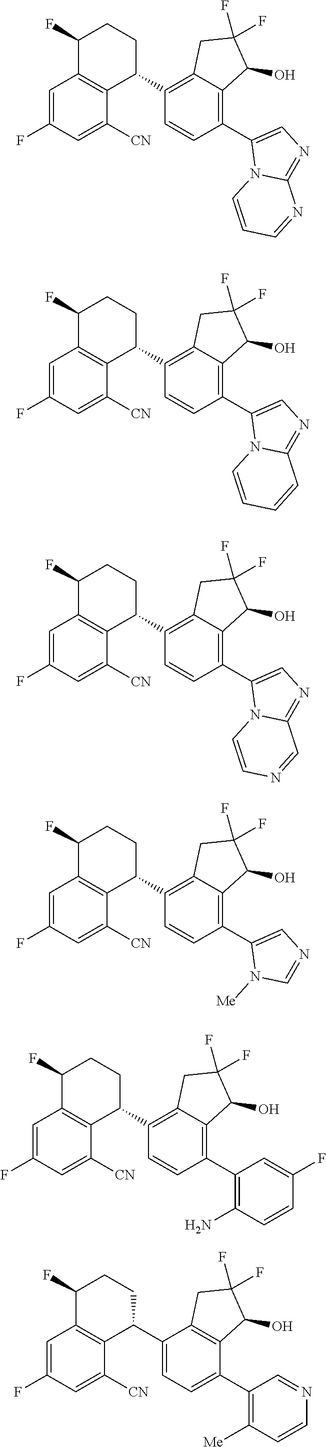

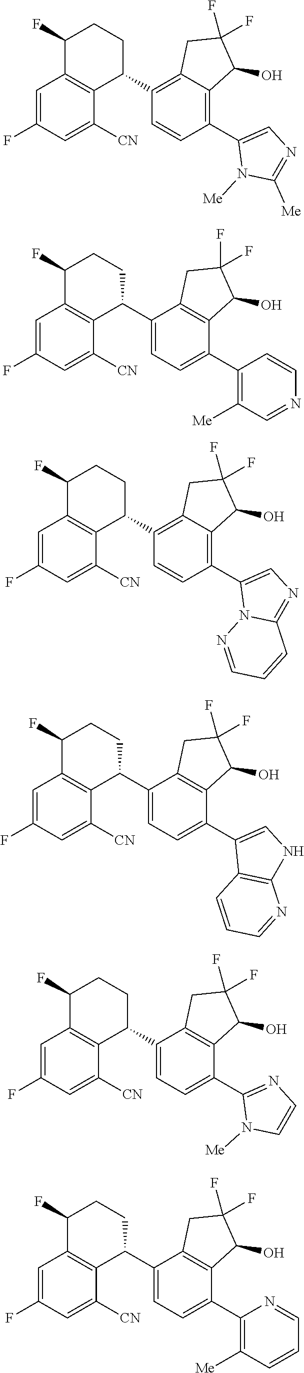

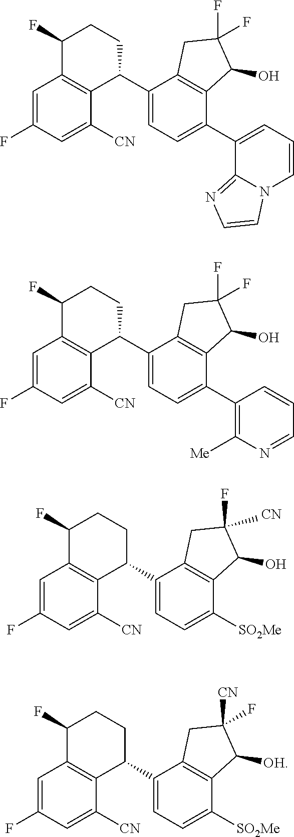

52. In embodiment 52, the method of any one of embodiments 1 to 51, is wherein the bladder cancer is selected from the group consisting of metastatic bladder cancer, non-metastatic bladder cancer, early-stage bladder cancer, non-invasive bladder cancer, muscle-invasive bladder cancer (MIBC), non-muscle-invasive bladder cancer (NMIBC), primary bladder cancer, advanced bladder cancer, locally advanced bladder cancer, bladder cancer in remission, progressive bladder cancer, recurrent bladder cancer and urothelial cancer.

53. In embodiment 53, the method of embodiment 52, is wherein the bladder cancer is muscle invasive bladder cancer.

54. In embodiment 54, the method of any one of embodiments 1 to 53, is wherein the method further comprises administering the HIF-2α inhibitor in combination with one or more additional anti-cancer agent, radiation therapy and/or surgery.

55. In embodiment 55, the method of any one of embodiments 1 to 54, is wherein the HIF-2α inhibitor and the one or more additional anti-cancer agent are administered sequentially or simultaneously.

56. In embodiment 56, the method of any one of embodiments 1 to 55, is wherein the the one or more anti-cancer agent is selected from the group consisting of vofatamab, infigratinib, LY2874455, pemigatinib, rogaratinib, PRN1371, zoligratinib, derazantinib, erdafitinib, nivolumab, pembrolizumab, pidilizumab, MEDI-0680, durvalumab, BMS-936559, cetrelimab, avelumab, atezolizumab, lapatinib, erlotinib, bevacizumab, sorafenib, carbonzanitib, pazopanib, olaparib, AZD1775, vistusertib, Linrodostat, Cisplatin, Carboplatin, Doxorubicin, Enfortumab Vedotin-ejfv, Mitomycin, Thiotepa, Sacituzumab Govitecan-hziy, valrubicin, gemcitabine, methotrexate, vinblastine, docetaxel, paclitaxel, pemetrexed, Bacillus Calmette-Guerin, and Interferon.

### Pharmaceutical Compositions

In general, the HIF-2α of this disclosure will be administered in a therapeutically effective amount by any of the accepted modes of administration for agents that serve similar utilities. The therapeutically effective amounts of HIF-2α inhibitors disclosed herein may range from about 100 mg to about 500 mg/per day, preferably 200 mg to 500 mg/day, which can be administered in single or multiple doses.

The actual amount of HIF-2α inhibitor, i.e., the active ingredients, will depend upon numerous factors such as the severity of the disease to be treated, the age and relative health of the patient, the potency of the compound being utilized, the route and form of administration, and other factors.

In general, the HIF-2α inhibitor of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a HIF-2α inhibitor of this disclosure in combination with at least one pharmaceutically acceptable excipient. Acceptable excipients are non-toxic, aid administration, and do not adversely affect the therapeutic benefit of the HIF-2α inhibitor. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

The level of the compound in a formulation can vary within the full range employed by those skilled in the art. Typically, the formulation will contain, on a weight percent (wt. %) basis, from about 0.01-99.99 wt. % of a HIF-2α inhibitor based on the total formulation, with the balance being one or more suitable pharmaceutical excipients. For example, the HIF-2α inhibitor is present at a level of about 1-80 wt. %.

The HIF-2α inhibitors disclosed herein can be administered either alone or in combination with one or more other anti-cancer drugs that are useful in the treatment of cancers for which compounds of this disclosure have utility. Such other drug(s) may be administered, by a route and in an amount commonly used therefore, simultaneously or sequentially with the HIF-2α inhibitor. It is also contemplated that when used in combination with such one or more other active ingredients, the HIF-2α inhibitor and the other active ingredients may be used in lower doses than when each is used singly.

Accordingly, the pharmaceutical compositions of the present disclosure also include those that contain one or more other drugs, in addition to HIF-2α inhibitor(s). The weight ratio of the compounds of this disclosure to the such other active ingredient may be varied and will depend upon the effective dose of each ingredient. Generally, an effective dose of each will be used.

Examples of such other anti-cancer agents include, but are not limited to, gossypol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec™), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™, such as Taxotere™.

Other anti-cancer agents include inhibitors of kinases associated cell proliferative disorder. These kinases include, but not limited to, Aurora-A, BTK, CDK1, CDK2, CDK3, CDK4, CDK6, CDK5, CDK7, CDK8, CDK9, ephrin receptor kinases, CHK1, CHK2, SRC, Yes, Fyn, Lck, Fer, Fes, Syk, Itk, Bmx, GSK3, JNK, MEK, PAK1, PAK2, PAK3, PAK4, PDK1, PKA, PKC, RAF, Rsk and SGK. In particular, inhibitors of CDK4/6, including abemaciclib (Verzenio), palbociclib (Ibrance) and ribociclib (Kisqali), have the potential to be synergistic with HIF-2α inhibitors and reverse the resistance to HIF-2α inhibition; mitogen-activated protein kinase signaling, e.g., U0126, PD98059, PD184352, PD0325901, ARRY-142886, SB239063, SP600125, BAY 43-9006, wortmannin, or LY294002; Syk inhibitors; antibodies (e.g., rituxan); MET inhibitor such as foretinib, carbozantinib, or crizotinib; VEGFR inhibitor such as sunitinib, sorafenib, regorafinib, lenvatinib, vandetanib, carbozantinib, axitinib; EGFR inhibitor such as afatinib, brivanib, carbozatinib, erlotinib, gefitinib, neratinib, lapatinib; PI3K inhibitor such as XL147, XL765, BKM120 (buparlisib), GDC-0941, BYL719, IPI145, BAY80-6946, BEX235 (dactolisib), CAL101 (idelalisib), GSK2636771, TG100-115; MTOR inhibitor such as rapamycin (sirolimus), temsirolimus, everolimus, XL388, XL765, AZD2013, PF04691502, PKI-587, BEZ235, GDC0349; MEK inhibitor such as AZD6244, trametinib, PD184352, pimasertinib, GDC-0973, AZD8330; CSF1R inhibitors (PLX3397, LY3022855, etc.) and CSF1R antibodies (IMC-054, RG7155, etc); TGF beta receptor kinase inhibitor such as LY2157299; BTK inhibitor such as ibrutinib.

Other anti-cancer agents include proteasome inhibitor such as carfilzomib, MLN9708, delanzomib, or bortezomib; BET inhibitors such as INCB054329, OTX015, CPI-0610; LSD1 inhibitors such as GSK2979552, INCB059872; HDAC inhibitors such as panobinostat, vorinostat; DNA methyl transferase inhibitors such as azacytidine, decitabine, and other epigenetic modulator; SHP-2 inhibitor such as TNO155; Bcl2 inhibitor ABT-199, and other Bcl-2 family protein inhibitors; HIF-2α inhibitors such as PT2977 and PT2385; Beta catenin pathway inhibitors, notch pathway inhibitors and hedgehog pathway inhibitors; Antibodies or other therapeutic proteins against VEGF include bevacizumab and aflibercept.

Other anti-cancer agents/drugs that can be used in combination with the compounds of the invention include, but are not limited to, liver X receptor (LXR) modulators, including LXR agonists and LXR beta-selective agonists; aryl hydrocarbon receptor (AhR) inhibitors.

Other anti-cancer agents that can be employed in combination with the compounds of this disclosure include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or Ril2), interferon alfa-2a; interferon alfa-2b; interferon alfa-n1; interferon alfa-n3; interferon beta-1a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with the compounds of the disclosure include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; Bfgf inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; R.sub.11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with the compounds of present disclosure include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomustine, etc.), or triazenes (decarbazine, etc.). Examples of antimetabolites include but are not limited to folic acid analog (e.g., methotrexate), or pyrimidine analogs (e.g., cytarabine), purine analogs (e.g., mercaptopurine, thioguanine, pentostatin).

Examples of natural products useful in combination with the compounds of this disclosure include but are not limited to vinca alkaloids (e.g., vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination the compounds of this disclosure include, but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to, folic acid analog (e.g., methotrexate), or pyrimidine analogs (e.g., fluorouracil, floxuridine, cytarabine), purine analogs (e.g., mercaptopurine, thioguanine, pentostatin.

Examples of hormones and antagonists useful in combination the compounds of this disclosure include, but are not limited to, adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Other anti-cancer agents that can be employed in combination with the compounds of the disclosure include: anti-cancer agents which act by arresting cells in the G2-M phases due to stabilized microtubules and include Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B. Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-TAABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

One or more additional immune checkpoint inhibitors can be used in combination with the compounds of this disclosure. Exemplary immune checkpoint inhibitors include inhibitors (smack molecules or biologics) against immune checkpoint molecules such as CD27, CD28, CD40, CD122, CD96, CD73, CD39, CD47, OX40, GITR, CSF1R, JAK, PI3K delta, PI3K gamma, TAM, arginase, CD137 (also known as 4-1BB), ICOS, A2AR, A2BR, SHP-2, B7-H3, B7-H4, BTLA, CTLA-4, LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, OX40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MED14736, MPDL3280A (also known as RG7446), or MSB0010718C. In some embodiments, the anti-PD-L1 monoclonal antibody is MPDL3280A (atezolizumab) or MED14736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of OX40, e.g., an anti-OX40 antibody or OX40L fusion protein. In some embodiments, the anti-OX40 antibody is MED10562 or, INCAGN01949, GSK2831781, GSK-3174998, MOXR-0916, PF-04518600 or LAG525. In some embodiments, the OX40L fusion protein is MEDI6383.

In addition, the combination therapy disclosed herein can be administered along with radiation.

### EXAMPLES

### Example 1

### Anti-Tumor Effect of Compound 1 in Mouse Bladder Cancer Xenograft Models

Compound 1 was formulated with 0.5% methylcellulose. 4˜10×10⁶ tumor cells in PBS and Corning® Matrigel (11 in volume) were inoculated subcutaneously at right flank for each Balb/c Nude mouse at 6-7 weeks of age for tumor development. Once tumors were 100-200 mm³ in size, the tumor bearing mice were randomly grouped into 2 groups (n=8) and treatment started with vehicle bid and Compound 1 at 20 mg/kg bid, respectively, for 17-30 days. Tumor sizes were measured twice weekly in two dimensions using a caliper and the volumes were expressed in mm³ using the formula V=0.5×a×b² where a and b were the long and short diameters of the tumor, respectively. All data were displayed as Mean and the standard error of the mean (SEM). As shown in FIG. 1, there is marginal anti-tumor effect in RT112 xenograft model, significant tumor growth inhibition in RT4 xenograft model, and complete tumor regression in T24 xenograft model by Compound 1 treatment.

### Example 2

### Induction of HIF-1α and HIF-2α Protein Levels Under Hypoxia Condition

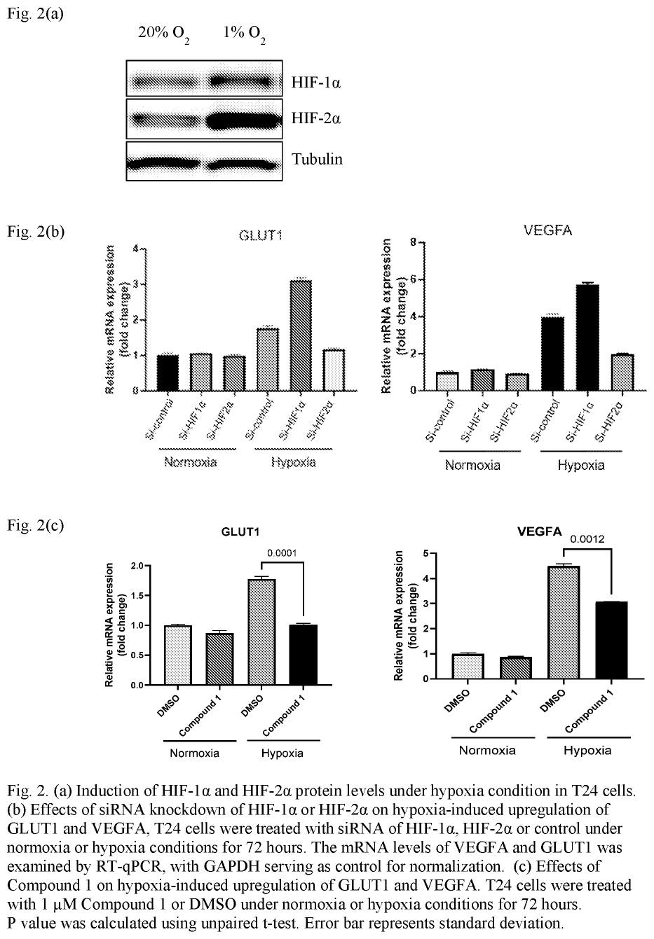

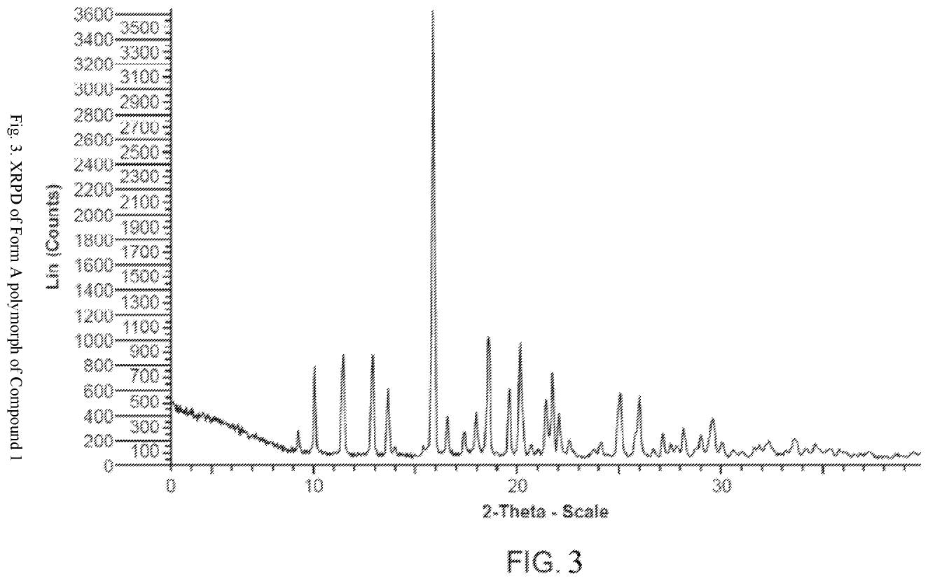

T24 cells were cultured in cell culture incubator with either 20% O₂ (normoxia) or 1% O₂ (hypoxia) for 72 hours. Cells were then rinsed with PBS and lysed in RIPA buffer containing Pierce Protease and phosphatase inhibitor (Thermo Scientific; catalog A32959). Protein concentration was quantified with the Pierce BCA Protein Assay kit (Thermo Scientific; catalog 23225). Isolated proteins were resolved by SDS-PAGE gels, transferred to nitrocellulose membrane (Thermo Scientific; catalog 1B23001), and detected by immunoblotting using the following antibodies: anti-HIF-1α (Cell signaling Technology; catalog 36169), anti-HIF-2α (Cell Signaling Technology; catalog 7096) and anti-tubulin (Cell Signaling Technology; catalog 15115). Both HIF-1α and HIF-2α could be detected under normoxia condition (20% O₂) and were upregulated under hypoxic condition (1% O₂) in T24 cells (FIG. 2 (a)).

### Example 3

### HIF-2α Inhibition by Compound 1 or siRNA Knockdown Suppressed the Hypoxia-Induced mRNA Upregulation of VEGFA and GLUT1

T24 cells were treated with DMSO or 1.0 μM of Compound 1; control siRNA or siRNA specific for HIF-1α, HIF-2α, for 72 h under normoxic (20% O₂) or hypoxic (1% O₂) conditions. Total RNA was extracted using RNeasy mini kit (Qiagen, Catalog #74104) and QIAshredder (Qiagen, Catalog #79654). Then RNA was reverse-transcribed into cDNA using Applied Biosystems™ High-Capacity cDNA Reverse Transcription Kit (Applied Biosystems™ cat #4368814). The cDNA was used as templates to examine the mRNA expression of VEGFA and GLUT1 by quantitative polymerase chain reaction (PCR) assays (PowerUp SYBR Green Master Mix by Applied Biosystems™, cat #A25742), with GAPDH serving as control for normalization. HIF-2α knockdown and Compound 1 treatment suppressed VEGFA and GLUT1 mRNA levels that were induced by hypoxia condition (FIGS. 2(b) and (c)). In contrast, HIF-1α knockdown upregulated VEGFA and GLUT1 mRNA levels (FIG. 2(b)).

What is claimed:

1. A method of treating bladder cancer in a patient, comprising administering to the patient in need thereof, a therapeutically effective amount of a HIF-2α inhibitor of Formula (I): 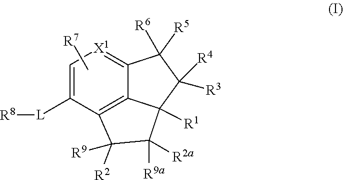
wherein:
X¹ is CH or N;
R¹ is hydroxy, halo, amino, —OP(O)(OH)₂, —OCH₂OP(O)(OH)₂, —OCOR¹⁰, —OCOOR¹¹, —OCONR¹²R¹³, —OCHR¹⁴OCOR¹⁵ or —OCHR¹⁴OCOOR^{15a} where R¹⁰, R¹¹, and R¹⁵ and R^{15a} are independently alkyl or alkyl substituted with amino, carboxy or hydroxy, R¹² and R¹³ are independently hydrogen, alkyl, or alkyl substituted with amino, carboxy or hydroxy or R¹² and R¹³ together with the nitrogen atom to which they are attached form optionally substituted heterocyclyl, and each R¹⁴ is hydrogen, alkyl, or haloalkyl;
R² is hydrogen, deuterium, alkyl, haloalkyl, alkenyl, or alkynyl;
R^{2a} is hydrogen or deuterium;
R³ and R⁴ are independently hydrogen, deuterium, alkyl, cycloalkyl, halo, haloalkyl, hydroxyalkyl, or alkoxyalkyl; or
R³ and R⁴ together with the carbon to which they are attached form oxo, 3 to 6 membered cycloalkylene, or 4 to 6 membered optionally substituted heterocyclylene;
R⁵ is hydrogen, deuterium, alkyl, halo, haloalkyl, hydroxy, or alkoxy;
R⁶ is hydrogen, deuterium, alkyl, cycloalkyl, or halo; or
R⁵ and R⁶ together with the carbon to which they are attached form oxo, alkyldienyl, 3 to 6 membered cycloalkylene, or 4 to 6 membered optionally substituted heterocyclylene; provided R⁵ and R⁶ and R³ and R⁴ together with the carbon to which they are attached do not form oxo, cycloalkylene or optionally substituted 4 to 6 membered heterocyclylene simultaneously;
R⁷ is hydrogen, deuterium, alkyl, alkoxy, cyano, halo, haloalkyl, or haloalkoxy;
L is a bond, S, SO, SO₂, O, CO, or NR¹⁶ where R¹⁶ is hydrogen or alkyl;
R⁸ is alkyl, haloalkyl, hydroxyalkyl, alkoxyalkyl, aminoalkyl, cycloalkyl, cycloalkenyl, bicyclic cycloalkyl, oxocycloalkenyl, cycloalkylalkyl, aryl, aralkyl, heterocyclyl, spirocycloalkyl, spiroheterocyclyl, heterocyclylalkyl, heteroaryl, or heteroaralkyl wherein aryl or heteroaryl, each by itself or as part of aralkyl or heteroaralkyl, or heterocyclyl by itself or as part of heterocyclylalkyl is substituted with R^{a}, R^{b}, R^{c}, R^{g} and R^{h} wherein R^{a}, R^{b}, and R^{c} are independently selected from hydrogen, deuterium, alkyl, haloalkyl, haloalkyloxy, alkoxy, hydroxy, halo, cyano, hydroxyalkyl, alkoxyalkyl, aminoalkyl, alkenyl, alkynyl, alkylidenyl, optionally substituted aryl, optionally substituted heteroaryl, and optionally substituted heterocyclyl and R^{g} and R^{h} are independently selected from hydrogen, deuterium, and halo;
R⁹ is hydrogen, alkyl, cycloalkyl, hydroxy, alkoxy, cyano, halo, haloalkyl, haloalkoxy, alkylsulfoxide, alkylsulfonyl, or heteroaryl wherein the heteroaryl is optionally substituted with R^{d}, R^{e}, and R^{f} independently selected from hydrogen, alkyl, haloalkyl, haloalkoxy, alkoxy, hydroxy, halo, and cyano; or
when R⁹ and R² are attached to the same carbon atom, they can combine to form oxo, alkyldienyl, 3 to 6 membered cycloalkylene, or 4 to 6-membered heterocyclylene; and
R^{9a} is hydrogen or deuterium;
or a pharmaceutically acceptable salt thereof.

2. The method of claim 1, wherein the compound of Formula (I) or a pharmaceutically acceptable salt thereof, has the structure of formula (IIa) or (IIb):

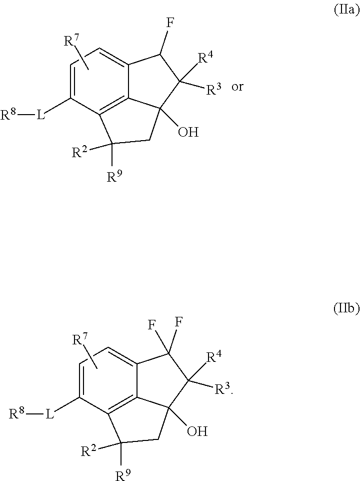

3. The method of claim 1, wherein the compound of Formula (I) or a pharmaceutically acceptable salt thereof, has the structure of formula (IIa′) or (IIb′):

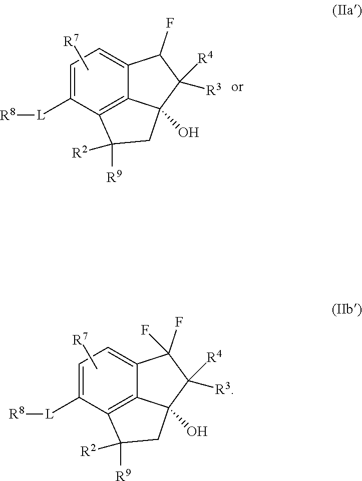

4. The method of any one of claims 1-3, wherein R³ is fluoro.

5. The method of any one of claims 1-3, where R³ and R⁴ are fluoro.

6. The method of any one of claims 1 to 5, wherein L is O.

7. The method of any one of claims 1 to 6, wherein R⁸ is phenyl substituted with R^{a}, R^{b}, R^{c}, R^{g} and R^{h} wherein R^{a}, R^{b}, and R^{c} are independently selected from hydrogen, deuterium, alkyl, haloalkyl, haloalkyloxy, alkoxy, hydroxy, halo, cyano, hydroxyalkyl, alkoxyalkyl, aminoalkyl, optionally substituted aryl, optionally substituted heteroaryl, and optionally substituted heterocyclyl and R^{g} and R^{h} are independently selected from hydrogen, deuterium, and halo.

8. The method of claim 7, wherein R⁸ is 3-chloro-5-fluorophenyl, 3,5-difluorophenyl, 3-fluoro-5-methoxyphenyl, 3-cyano-5-fluorophenyl, 3-chloro-5-cyanophenyl, 3-cyano-5-methylphenyl, 3-chloro-4-fluorophenyl, 3-chloro-5-fluorophenyl, 3-fluoro-5-methyphenyl, 3-cyanophenyl, 3-trifluoromethylphenyl, 3,4-dichlorophenyl, 3-chloro-2-methylphenyl, 3,5-dichlorophenyl, 3,5-dimethylphenyl, 2-chloro-6-methylphenyl, 2,6-difluorophenyl, 3,4,5-trifluorophenyl, 3,4-difluorophenyl, 4-fluoro-3-methylphenyl, 3-cyano-4-fluorophenyl, 3-cyano-5-difluoromethylphenyl or 3-cyano-5-fluoro-2,4,6-trideuteriophenyl.

9. The method of any one of claims 1 to 8, wherein
R⁷ is hydrogen;
R² is hydrogen, methyl, or ethyl; and
R⁹ is hydrogen, alkyl, fluoro, hydroxy, or alkoxy.

10. The method of claim 1, wherein the HIF-2α inhibitor of Formula (I) is selected from:
3-fluoro-5-((3,3,4,4-tetrafluoro-2a-hydroxy-1-methylene-2,2a,3,4-tetrahydro-1H-cyclopenta[cd]inden-7-yl)oxy)benzonitrile;
3-fluoro-5-((3,3,4,4-tetrafluoro-2a-hydroxy-1-oxo-2,2a,3,4-tetrahydro-1H-cyclopenta[cd]inden-7-yl)oxy)benzonitrile;
3-fluoro-5-((3,3,4,4-tetrafluoro-1,2a-dihydroxy-2,2a,3,4-tetrahydro-1H-cyclopenta[cd]inden-7-yl)oxy)benzonitrile;
3-fluoro-5-((1,3,3,4,4-pentafluoro-2a-hydroxy-2,2a,3,4-tetrahydro-1H-cyclopenta[cd]inden-7-yl)oxy)benzonitrile;
3-fluoro-5-(((1S,2aR)-1,3,3,4,4-pentafluoro-2a-hydroxy-2,2a,3,4-tetrahydro-1H-cyclopenta[cd]inden-7-yl)oxy)benzonitrile;
3-fluoro-5-(((1R,2aS)-1,3,3,4,4-pentafluoro-2a-hydroxy-2,2a,3,4-tetrahydro-1H-cyclopenta[cd]inden-7-yl)oxy)benzonitrile;
3-fluoro-5-(((1R,2aR)-1,3,3,4,4-pentafluoro-2a-hydroxy-2,2a,3,4-tetrahydro-1H-cyclopenta[cd]inden-7-yl)oxy)benzonitrile;
1,3,3,4,4-pentafluoro-7-((5-fluoropyridin-3-yl)oxy)-1,2,3,4-tetrahydro-2aH-cyclopenta[cd]inden-2a-ol;
3-fluoro-5-((3,3,4,4-tetrafluoro-2a-hydroxy-2,2a,3,4-tetrahydrospiro[cyclopenta[cd]-indene-1,1′-cyclopropan]-7-yl)oxy)benzonitrile;
3-fluoro-5-((3,3,4,4-tetrafluoro-2a-hydroxy-1-methyl-2,2a,3,4-tetrahydro-1H-cyclopenta[cd]inden-7-yl)oxy)benzonitrile;
3-fluoro-5-((3,3,4,4-tetrafluoro-1,2a-dihydroxy-1-methyl-2,2a,3,4-tetrahydro-1H-cyclopenta[cd]inden-7-yl)oxy)benzonitrile;
3-fluoro-5-((1,3,3,4,4-pentafluoro-2a-hydroxy-1-methyl-2,2a,3,4-tetrahydro-1H-cyclopenta[cd]inden-7-yl)oxy)benzonitrile;
3-fluoro-5-((3,3,4,4-tetrafluoro-2a-hydroxy-2,2a,3,4-tetrahydro-1H-cyclopenta[cd]inden-7-yl)oxy)benzonitrile;
3-((2a-amino-1,3,3,4,4-pentafluoro-2,2a,3,4-tetrahydro-1H-cyclopenta[cd]inden-7-yl)oxy)-5-fluorobenzonitrile;
3-fluoro-5-(((1S,2aR)-1,3,3,4,4-pentafluoro-2a-hydroxy-2,2a,3,4-tetrahydro-1H-cyclopenta[cd]inden-7-yl-1,2,2-d3)oxy)benzonitrile;
3-fluoro-5-(((1S,2aR)-1,3,3,4,4-pentafluoro-2a-hydroxy-2,2a,3,4-tetrahydro-1H-cyclopenta[cd]inden-7-yl-1-d)oxy)benzonitrile-2,4,6-d3;
(R)-3-fluoro-5-((3,3,4,4-tetrafluoro-2a-hydroxy-1-methylene-2,2a,3,4-tetrahydro-1H-cyclopenta[cd]inden-7-yl)oxy)benzonitrile; and
(S)-3-fluoro-5-((3,3,4,4-tetrafluoro-2a-hydroxy-1-methylene-2,2a,3,4-tetrahydro-1H-cyclopenta[cd]inden-7-yl)oxy)benzonitrile; or
a pharmaceutically acceptable salt thereof.

11. The method of claim 1, wherein the HIF-2α inhibitor of Formula (I) is 3-fluoro-5-((1,3,3,4,4-pentafluoro-2a-hydroxy-2,2a,3,4-tetrahydro-1H-cyclopenta[cd]inden-7-yl)oxy)-benzonitrile.

12. The method of claim 1, wherein the HIF-2α inhibitor of Formula (I) is 3-fluoro-5-(((1S,2aR)-1,3,3,4,4-pentafluoro-2a-hydroxy-2,2a,3,4-tetrahydro-1H-cyclopenta[cd]inden-7-yl)oxy)-benzonitrile.

13. The method of claim 12, wherein the 3-fluoro-5-(((1S,2aR)-1,3,3,4,4-pentafluoro-2a-hydroxy-2,2a,3,4-tetrahydro-1H-cyclopenta[cd]inden-7-yl)oxy)-benzonitrile is a crystalline solid, designated as Form A polymorph, having an X-ray powder diffraction pattern comprising peaks at angular positions 15.8 and 18.6, wherein the angular positions may vary by ±0.2° 2θ as measured by X-ray powder diffraction using (Cu Ku) an X-ray wavelength of 1.5418 Å.

14. The method of claim 13, wherein the Form A polymorph X-ray powder diffraction pattern further comprises a peak at angular position 20.1, wherein the angular position may vary by ±0.2° 2θ.

15. The method of claim 13, wherein the Form A polymorph X-ray powder diffraction pattern further comprises peaks at angular positions 12.9 and 20.1, wherein the angular positions may vary by ±0.2° 2θ.

16. The method of claim 13, wherein the Form A polymorph X-ray powder diffraction pattern further comprises peaks at angular positions 11.4, 12.9, and 20.1, wherein the angular positions may vary by ±0.2° 2θ.

17. The method of claim 13, wherein the Form A polymorph X-ray powder diffraction pattern further comprises peaks at angular positions 10.1, 11.4, 12.9, and 20.1, wherein the angular positions may vary by ±0.2° 2θ.

18. The method of claim 12, wherein the 3-fluoro-5-(((1S,2aR)-1,3,3,4,4-pentafluoro-2a-hydroxy-2,2a,3,4-tetrahydro-1H-cyclopenta[cd]inden-7-yl)oxy)-benzonitrile is a crystalline solid, designated as Form A polymorph, having an X-ray powder diffraction pattern having an X-ray powder diffraction pattern substantially identical as shown in FIG. 1.

19. The method of any one of claims 13 to 17, wherein the angular positions of the X-ray powder diffraction pattern peaks denoted therein may vary by ±0.1° 2θ.

20. The method of any one of claims 1 to 19, wherein the bladder cancer is selected from the group consisting of metastatic bladder cancer, non-metastatic bladder cancer, early-stage bladder cancer, non-invasive bladder cancer, muscle-invasive bladder cancer (MIBC), non-muscle-invasive bladder cancer (NMIBC), primary bladder cancer, advanced bladder cancer, locally advanced bladder cancer, bladder cancer in remission, progressive bladder cancer, recurrent bladder cancer, and urothelial cancer.

21. The method of claim 20, wherein the bladder cancer is muscle invasive bladder cancer.

22. The method of any one of claims 1 to 21, wherein the method further comprises administering the HIF-2α inhibitor in combination with one or more additional anti-cancer agent, radiation therapy and/or surgery.

23. The method of any one of claims 1 to 22, wherein the HIF-2α inhibitor and the one or more additional anti-cancer agent are administered sequentially or simultaneously.

24. The method of claim 22 or 23, wherein the one or more anti-cancer agent is selected from the group consisting of vofatamab, infigratinib, LY2874455, pemigatinib, rogaratinib, PRN1371, zoligratinib, derazantinib, erdafitinib, Debio-1347, nivolumab, pembrolizumab, pidilizumab, MEDI-0680, durvalumab, BMS-936559, cetrelimab, avelumab, atezolizumab, afatinib, lapatinib, erlotinib, pertuzumab, trastuzumab, trastuzumab deruxtecan, bevacizumab, ramucirumab, sorafenib, carbonzanitib, lenvatinib, pazopanib, olaparib, AZD1775, vistusertib, Linrodostat, Cisplatin, Carboplatin, Doxorubicin, Enfortumab Vedotin-ejfv, Mitomycin, RC48-ADC, Thiotepa, Sacituzumab Govitecan-hziy, valrubicin, gemcitabine, methotrexate, vinblastine, docetaxel, paclitaxel, pemetrexed, Bacillus Calmette-Guerin, and Interferon.
